SEQUENCE LISTING (22 sequences)

SEQ ID NO: 1 - DNA, 18 bp, Artificial Sequence
  pER6 consensus sequence
  misc_feature (0)...(0): m = a or c k = g or t/u
  misc_feature 7, 8, 9, 10, 11, 12: n = A,T,C or G
  misc_feature 7, 8, 9, 10, 11, 12: n = A,T,C or G
  misc_feature 7, 8, 9, 10, 11, 12: n = A,T,C or G
tgamctnnnnnnagktca

SEQ ID NO: 2 - DNA, 31 bp, Artificial Sequence
  CYP3A4 pER6 oligomer
tagaatatgaactcaaaggaggtcagtgagt

SEQ ID NO: 3 - DNA, 31 bp, Artificial Sequence
  Complementary oligomer to CYP3A4 pER6 oligomer
actcactgacctcctttgagttcatattcta

SEQ ID NO: 4 - DNA, 15 bp, Artificial Sequence
  dDR3 enhancer consensus sequence
  misc_feature (0)...(0): M is A or C, Y is T or C
  misc_feature 7, 8, 9: n = A,T,C or G
  misc_feature 7, 8, 9: n = A,T,C or G
tgamcynnntgamcy

SEQ ID NO: 5 - DNA, 21 bp, Artificial Sequence
  CYP3A4 dDR3 oligomer
gaatgaacttgctgaccctct

SEQ ID NO: 6 - DNA, 21 bp, Artificial Sequence
  Complementary oligomer to CYP3A4 dDR3 oligomer
agagggtcagcaagttcattc

SEQ ID NO: 7 - DNA, 18 bp, Artificial Sequence
  dER6 enhancer consensus sequence
  misc_feature (0)...(0): M is A or C, Y is T or C, K is G or T, and R is G or A
  misc_feature 7, 8, 9, 10, 11, 12: n = A,T,C or G
  misc_feature 7, 8, 9, 10, 11, 12: n = A,T,C or G
tgaamynnnnnnkrttca

SEQ ID NO: 8 - DNA, 26 bp, Artificial Sequence
  CYP3A4 dER6 oligomer
cccttgaaatcatgtcggttcaagca

SEQ ID NO: 9 - DNA, 26 bp, Artificial Sequence
  Complementary oligomer to CYP3A4 dER6 oligomer
tgcttgaaccgacatgatttcaaggg

SEQ ID NO: 10 - DNA, 18 bp, Artificial Sequence
  CYP3A4 pER6 enhancer element
tgaactcaaaggaggtca

SEQ ID NO: 11 - DNA, 15 bp, Artificial Sequence
  CYP3A4 dDR enhancer element
tgaacttgctgaccc

SEQ ID NO: 12 - DNA, 18 bp, Artificial Sequence
  CYP dER6 enhancer element
tgaaatcatgtcggttca

SEQ ID NO: 13 - DNA, 120 bp, Cytomegalovirus
  misc_feature (0)...(0): CMV immediate early minimal promoter
taggcgtgtacggtgggaggcctatataagcagagctcgtttagtgaaccgtcagatcgcctggagacgccatccacgctgttttgacctccatagaagacaccgggaccgatccagcct

SEQ ID NO: 14 - DNA, 88 bp, Artificial Sequence
  Clone 26-SEAP promoter
tagaatatgaactcaaaggaggtcagtgagttagaatatgaactcaaaggaggtcagtgagtcccttgaaatcatgtcggttcaagca

SEQ ID NO: 15 - DNA, 151 bp, Artificial Sequence
  Clone 33-SEAP promoter
tgcttgaaccgacatgatttcaagggagagggtcagcaagttcattctagaatatgaactcaaaggaggtcagtgagtgaatgaacttgctgaccctctgaatgaacttgctgaccctctactcactgacctcctttgagttcatattcta

SEQ ID NO: 16 - DNA, 141 bp, Artificial Sequence
  Clone 61-SEAP promoter
agagggtcagcaagttcattcagagggtcagcaagttcattctgcttgaaccgacatgatttcaagggagagggtcagcaagttcattctagaatatgaactcaaaggaggtcagtgagtagagggtcagcaagttcattc

SEQ ID NO: 17 - DNA, 286 bp, Artificial Sequence
  Clone 71-SEAP promoter
tgcttgaaccgacatgatttcaagggcccttgaaatcatgtcggttcaagcaactcactgacctcctttgagttcatattctacccttgaaatcatgtcggttcaagcaagagggtcagcaagttcattctgcttgaaccgacatgatttcaagggtgcttgaaccgacatgatttcaagggagagggtcagcaagttcattcactcactgacctcctttgagttcatattctagaatgaacttgctgaccctctactcactgacctcctttgagttcatattcta

SEQ ID NO: 18 - DNA, 110 bp, Artificial Sequence
  Clone 71-SEAP promoter
agagggtcagcaagttcattctgcttgaaccgacatgatttcaagggagagggtcagcaagttcattcgaatgaacttgctgaccctctgaatgaacttgctgaccctct

SEQ ID NO: 19 - DNA, 33 bp, Artificial Sequence
  PCR primer
gaagcttgttcatggccagtagggaagatgagc

SEQ ID NO: 20 - DNA, 27 bp, Artificial Sequence
  PCR primer
tggcctcagctgcagatctcctggagc

SEQ ID NO: 21 - DNA, 5088 bp, Artificial Sequence
  pVIj-SEAP-polyEcoRV
agtgcaccatatgaacttcagctgacgctgatatcaatgcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcagcttcctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctccgcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaacccgtggtccccgcgttgcttcctctgctggccgggacatcaggtggcccccgctgaattggaatcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtccctggcggacgggaagtatcagctcgaccaagctctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgag

SEQ ID NO: 22 - DNA, 1801 bp, Artificial Sequence
  Clone 102-SEAP reporter gene cassette
agagggtcagcaagttcattctgcttgaaccgacatgatttcaagggagagggtcagcaagttcattcgaatgaacttgctgaccctctgaatgaacttgctgaccctctatcaatgcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcggccccgaattcagcttcctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctccgcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaa

(19) United States
(12) Patent Application Publication
(10) Pub. No.: US 2007/0264674 A1
(43) Pub. Date: Nov. 15, 2007

(54) Cytochrome P450 Induction Assay

(76) Inventors: Giacomo Paonessa, Pomerzia (IT); Sandra Cicuzza, Pomezia (IT); Ralph Laufer, Pomezia (IT)

Correspondence Address: MERCK AND CO., INC, P O BOX 2000, RAHWAY, NJ, 07065-0907, US

(21) Appl. No.: 11/660,721

(22) PCT Filed: Aug. 15, 2005
(86) PCT No.: PCT/EP05/08864

§ 371 Date: Feb. 20, 2007

(30) Foreign Application Priority Data
Aug. 20, 2004 (IT) 60603163

(51) Int. Cl. G01N 33/53; G01N 33/569

(52) U.S. Cl. 435/7.21; 435/7.2

(57) ABSTRACT

A method for identifying compounds that can induce expression of cytochrome P450, in particular, expression of the CYP3A4 isoform, is described. The method provides a reporter gene operably linked to a composite promoter comprising in tandem one or more cis-acting elements, which are bound by activated pregnane X receptor (PXR), operably linked to a heterologous promoter. Analytes, which are inducers CYP3A4 expression via PXR activation, induce expression of the reporter gene.

### BACKGROUND OF THE INVENTION

(1) Field of the Invention

The present invention relates to a method for identifying analytes that can induce expression of cytochrome P450, in particular, expression of the CYP3A4 isoform. The method provides a reporter gene operably linked to a composite promoter comprising in tandem one or more cis-acting elements, which are bound by activated pregnane X receptor (PXR), operably linked to a heterologous promoter. Analytes, which are inducers CYP3A4 expression via PXR activation, induce expression of the reporter gene.

(2) Description of Related Art

Cytochrome P450 (CYPs) are involved in the hydroxylation of many drugs, carcinogens, pesticides and xenobiotics and many CYPs are strongly inducible by xenobiotics, up to 50 to 100 fold. In drug therapy, there are two major concerns with respect to CYP induction. First, induction may cause a reduction in therapeutic efficacy by decreasing systemic exposure as a result of increased drug metabolism. Second, induction may create an undesirable imbalance between toxification and detoxification as a result of increased formation of reactive metabolites (Lin and Lu, Clin. Pharmacokinet. 35: 361-390 (1998)).

Although many of the CYP enzymes are known to be inducible, CYP3A4 induction is probably the most important cause for the documented induction-based drug-CYP interactions (Whitlock et al., In Cytochrome P450: Structure, Mechanism and Biochemistry (Second edition). Ortiz de Montellano (Ed.). Plenum Press, New York (1995). pp. 367-390). This is because CYP3A4 accounts for roughly 40% of the total CYP in human liver and it metabolizes more than 60% of clinically used drugs. Because a drug candidate may have the potential for inducing undesirable CYP3A4 induction-based interactions, it has become important to assess new drug candidates for their CYP3A4 induction potential.

Although in vivo animal models may provide some useful information on the factors that affect the in vitro/in vivo extrapolation of induction data, significant species differences in the inductive response preclude the use of animal models for the assessment of human CYP3A4 induction for new drug candidates. Several in vitro models have been established to assess the potential of CYP3A4 induction for new drug candidates, including liver slices, immortalized cell lines, and primary hepatocytes (Silva et al., Drug Metab. Disp. 26: 490-496 (1998); Kostrubsky et al., Drug Metab. Disp. 27: 887-894 (1999); Maurel, Adv. Drug Dev. Rev. 22:105-132 (1996); LeCluyse, Eur. J. Pharma. Sci. 13: 343-368 (2001)). Among these models, primary cultures of human hepatocytes have been used extensively by academic and industrial laboratories for evaluating CYP3A4 induction. It is generally believed that the primary hepatocyte culture is the most predictive in vitro model for assessing CYP induction; however, the availability of human hepatocytes is often very limited. Therefore, the use of in vitro systems is the only means by which the potential of human CYP3A4 induction can be assessed in a high-throughput screening mode.

Since the publication of the nucleotide sequence of its proximal region (Hashimoto et al., Eur. J. Biochem. 218: 585-595 (1993)), the promoter of the CYP3A4 gene has been analyzed in detail for cis-acting elements that confer responsiveness to xenobiotics. Several important elements have been identified. A proximal element, prPXRE (proximal ER6 or pER6), contains two copies of a TGA(A/C)CT hexamer motif, the recognition sequence for the nuclear receptor family of transcription factors (Mangelsdorf et al., Cell 83: 835-839 (1995)), organized as an ER6 (everted repeat separated by six nucleotides). prPXRE confers relatively modest activation by rifampicin of reporter gene expression (Barwick et al., Mol. Pharmacol. 50: 10-16 (1996); Ogg et al., Xenobiotica 29: 269-279 (1999)). The prPXRE enhancer element has relatively low activation in response to rifampicin. However, Goodwin et al., (Mol. Pharmacol. 56: 1329-1339 (1999)) and Liddle and Goodwin (WO 9961622) identified a 230 bp distal element called the xenobiotic-responsive enhancer module (XREM) located about 8 kb upstream from the CYP3A4 transcription start site which they characterized as a potent enhancer of the CYP3A4 by activators of human PXR activity. The XREM contains two nuclear receptor (NR) binding sites, dNR1 (distal DR3 or dDR3) and dNR2 (distal ER6 or dER6), separated by 29 nucleotides. The dNR1 (distal DR3 or dDR3) element contains two copies of a TGA(A/C)C(T/C) hexamer organized as a direct repeat separated by three nucleotides. The dNR2 (distal ER6 or dER6) element contains two copies of a TGAA(A/C)(T/C) hexamer organized as an everted repeat separated by six nucleotides. Sueyoshi and Negishi (Annu. Rev. Pharmacol. Toxicol. 41: 123-143 (2001) provide a review of enhancer elements in the CYP3A family.

The regulation of the human CYP3A4 promoter has been found to be very complex. Many human nuclear receptors, for example, pregnane X receptor (PXR), constitutive androgen receptor (CAR), retinoid X receptor (RXR), vitamin D receptor (VDR), glucocorticoid receptor, and transcriptional factors are involved in the regulation of CYP3A4 transcriptional activity (Pascussi et al., Biochim. Biophys. Acta 1619, 243-253 (2003)), but among these nuclear receptors, PXR together with RXR seem to play a major role in regulating CYP3A4 transcriptional activity (See WO9935246 to Evans and Blumberg which discloses binding of PXR to RXR.). The PXR is activated by a variety of lipophilic compounds, many of which, such as Rifampicin and other drugs, are known CYP3A inducers (Bertilsson et al., Proc. Natl. Acad. Sci. USA 95: 12208-12213 (1998); Blumberg et al., Genes Dev. 12: 3195-3205 (1998); Lehmann et al., J. Clin. Invest. 102: 1016-1023 (1998)). WO9935246 to Evans and Blumberg, WO 9948915 to Kliewer and Willson and Kliewer et al., Cell 92: 73-82 (1998) disclose the nucleotide sequence for the human PXR and methods for using the human PXR in assays to screen compounds for its ability to activate or inhibit human PXR. Nucleotide sequences encoding the human CAR has been disclosed in U.S. Pat. No. 6,579,686 to Collins and Park and WO9317041 to Moore and Baes discloses a polypeptide which appears related to the CAR.

Cell lines such as HepG2 normally express high amounts of RXR but very little PXR. Transfection of the CYP3A4 promoter (whole or part of it) fused to reporter genes in HepG2 cells results in clear induction by Rifampicin but only it is co-transfected with human PXR (Blumberg et al., Genes Dev. 12: 3195-3205 (1998); Goodwin et al., Mol. Pharmacol. 56:1329-1339 (1999)). These findings led to the development of PXR reporter gene assays for screening the induction potential of drugs (Moore et al., J. Biol. Chem. 275: 15122-15127 (2000); E1-Sankary et al., Drug Metab. Disp. 29: 1499-1504 (2001); Luo et al., Drug Metab. Disp. 30: 795-804 (2002); Drocourt et al., Drug Metab. Disp., 29, 1325-1331 (2001)).

Electrophoretic Mobility Shift Assays (EMSA) have shown that the human PXR together with human RXR bind directly to CYP3A4 promoter sequence motifs prPXRE, dNR1, and dNR2 (Blumberg et al., Genes Dev. 12: 3195-3205 (1998); Lehmann et al., J. Clin. Invest. 102: 1016-1023 (1998); Goodwin et al., Mol. Pharmacol. 56:1329-1339 (1999)). For example, when an expression vector consisting of nucleotides −7839 to −7208 and −362 to +64 of the CYP3A4 promoter (ΔCYP3A4 promoter) was operably linked to a reporter gene and the expression vector transfected into HepG2 cells with an human PXR expression vector, induction of expression of the reporter gene was observed upon Rifampicin treatment (Goodwin et al., Mol. Pharmacol. 56:1329-1339 (1999); WO 9961622 to Liddle and Goodwin). Recently, similar EMSA experiments also demonstrated that human CAR binds directly to both prPXRE and dNR1 and to a lesser extent to dNR2, demonstrating the induction potential of CYP3A4 by CAR ligands (Goodwin et al., Mol. Pharmacol. 62:359-3652002).

The above human PXR reporter assays have been useful for screening drugs for their CYP induction potential. However, the above assays may not detect drugs which cause CYP induction at a low level. Therefore, there is a need for an assay that is capable of providing an induction response that will enable drugs with a low level of CYP inducibility to be detected.

### BRIEF SUMMARY OF THE INVENTION

The present invention provides a composite promoter comprising in tandem one or more cis-acting elements, which are bound by an activated pregnane X receptor (PXR), operably linked to a heterologous promoter, and methods for using the composite promoter operably linked to a reporter gene to identify analytes that can induce expression of cytochrome P450, in particular, expression of the CYP3A4 isoform. Analytes, which are inducers CYP3A4 expression via PXR, induce expression of the reporter gene.

Thus, the present invention provides a composite promoter comprising a nucleic bearing two or more PXR binding sites in tandem operably linked to a nucleic acid bearing a heterologous promoter, which is not inducible by PXR in the absence of the nucleic acid comprising the PXR binding sites. That is a composite promoter wherein one or more nucleic acids, each bearing a PXR binding site, are operably linked to a nucleic acid bearing a heterologous promoter, preferably a heterologous promoter that is not PXR-inducible in the absence of the PXR binding sites. Preferably, when there are more two or more nucleic acids, each comprising a PXR binding site, the nucleic acids are linked in tandem to the heterologous promoter. The PXR binding sites operably linked to the heterologous promoter render the heterologous promoter responsive to PXR induction of transcription from the promoter.

In a further embodiment of the above promoter, the PXR binding sites are selected from the group consisting of dDR3, dER6, and pER6. In further still embodiments, the dDR3 comprises the nucleotide sequence of SEQ ID NO:4, the dER6 comprises the nucleotide sequence of SEQ ID NO:7, and the pER6 comprises the nucleotide sequence of SEQ ID NO:1, or, the dDR3 comprises the nucleotide sequence of SEQ ID NO:5, the dER6 comprises the nucleotide sequence of SEQ ID NO:8, and the pER6 comprises the nucleotide sequence of SEQ ID NO:2, or, the dDR3 comprises the nucleotide sequence of SEQ ID NO:11, the dER6 comprises the nucleotide sequence of SEQ ID NO:12, and the pER6 comprises the nucleotide sequence of SEQ ID NO:10.

In a further still embodiment of the above promoter, the above composite promoter comprises at least one of the dER6 binding sites and at least one of the dDR3 binding sites, at least one of the dER6 binding sites and at least one of the pER6 binding sites, at least three of the dDR3 binding sites, or three of the dDR3 binding sites and one each of the dER6 and pER6 binding sites. In further still embodiments, the composite promoter comprises at least three dDR3 sites. In a particularly preferred embodiment of the above composite promoter, the nucleic acid bearing the two or more PXR binding sites in tandem comprises the nucleotide sequence of SEQ ID NO:18. In a further embodiment, the composite promoter comprises SEQ ID NO:18 operably linked to the CMV promoter comprising SEQ ID NO:13.

The present invention further provides a reporter gene expression cassette comprising a nucleic acid, which includes two or more PXR binding sites in tandem operably linked to a heterologous promoter that is not inducible by PXR in the absence of the PXR binding sites, operably linked to a reporter gene.

In a further still aspect, the reporter gene expression cassette comprises a first nucleic acid which includes two or more PXR binding sites operably linked to a second nucleic acid bearing a heterologous promoter that is not inducible by PXR in the absence of the nucleic acid comprising the PXR binding sites operably linked to a third nucleic acid encoding a reporter gene.

Thus, the present invention provides a reporter gene expression cassette that includes a first nucleic acid comprising two or more nucleic acids, each bearing a PXR binding site, in tandem operably linked to a second nucleic acid bearing a heterologous promoter that is not inducible by PXR in the absence of the nucleic acid comprising the PXR binding sites, operably linked to a third nucleic acid encoding a reporter gene.

In a further embodiment of any one of the above reporter gene expression cassette, the PXR binding sites are selected from the group consisting of dDR3, dER6, and pER6. In further still embodiments, the dDR3 comprises the nucleotide sequence of SEQ ID NO:4, the dER6 comprises the nucleotide sequence of SEQ ID NO:7, and the pER6 comprises the nucleotide sequence of SEQ ID NO:1, or, the dDR3 comprises the nucleotide sequence of SEQ ID NO:5, the dER6 comprises the nucleotide sequence of SEQ ID NO:8, and the pER6 comprises the nucleotide sequence of SEQ ID NO:2, or, the dDR3 comprises the nucleotide sequence of SEQ ID NO:1, the dER6 comprises the nucleotide sequence of SEQ ID NO:12, and the pER6 comprises the nucleotide sequence of SEQ ID NO:10.

In a further still embodiment of the above reporter gene expression cassette, the first nucleic acid comprises at least one of the dER6 binding sites and at least one of the dDR3 binding sites, the nucleic acid comprises at least one of the dER6 binding sites and at least one of the pER6 binding sites, the nucleic acid comprises at least three of the dDR3 binding sites, or the nucleic acid comprises three of the dDR3 binding sites and one each of the dER6 and pER6 binding sites. In further still embodiments, the nucleic acid comprises at least three dDR3 sites. In a particularly preferred embodiment of the above reporter gene expression cassette, the nucleic acid comprising the two or more PXR binding sites in tandem comprises the nucleotide sequence of SEQ ID NO:18. In a further preferred embodiment, the reporter gene expression cassette comprises the reporter gene expression cassette of clone 102-SEAP.

In particular embodiments of the above reporter gene cassette, the reporter is secreted embryonic alkaline phosphatase (SEAP).

The present invention further provides a cell comprising a nucleic acid which includes two or more PXR binding sites in tandem operably linked to a heterologous promoter that is not inducible by PXR in the absence of the PXR binding sites operably linked to a reporter gene.

In a further aspect, the cell comprises a first nucleic acid which includes two or more PXR binding sites in tandem operably linked to a heterologous promoter that is not inducible by PXR in the absence of PXR binding sites operably linked to a reporter gene and a second nucleic acid which includes encodes the PXR operably linked to a heterologous promoter.

In a further aspect, the cell comprises a first nucleic acid which includes two or more PXR binding sites in tandem operably linked to a heterologous promoter that is not inducible by PXR in the absence of PXR binding sites operably linked to a reporter gene and a second nucleic acid which includes encodes a CAR operably linked to a heterologous promoter.

Thus, the present invention provides a cell comprising a reporter gene expression cassette that includes a first nucleic acid comprising two or more nucleic acids bearing PXR binding sites in tandem operably linked to a second nucleic acid bearing a heterologous promoter that is not inducible by PXR in the absence of the nucleic acid comprising the PXR binding sites, operably linked to a third nucleic acid encoding a reporter gene.

In a further embodiment of any one of the above cells, the PXR binding sites are selected from the group consisting of dDR3, dER6, and pER6. In further still embodiments, the dDR3 comprises the nucleotide sequence of SEQ ID NO:4, the dER6 comprises the nucleotide sequence of SEQ ID NO:7, and the pER6 comprises the nucleotide sequence of SEQ ID NO:1, or, the dDR3 comprises the nucleotide sequence of SEQ ID NO:5, the dER6 comprises the nucleotide sequence of SEQ ID NO:8, and the pER6 comprises the nucleotide sequence of SEQ ID NO:2, or, the dDR3 comprises the nucleotide sequence of SEQ ID NO:11, the dER6 comprises the nucleotide sequence of SEQ ID NO:12, and the pER6 comprises the nucleotide sequence of SEQ ID NO:10.

In a further still embodiment of the above cell, the first nucleic acid comprises at least one of the dER6 binding sites and at least one of the dDR3 binding sites, the nucleic acid comprises at least one of the dER6 binding sites and at least one of the pER6 binding sites, the nucleic acid comprises at least three of the dDR3 binding sites, or the nucleic acid comprises three of the dDR3 binding sites and one each of the dER6 and pER6 binding sites. In further still embodiments, the nucleic acid comprises at least three dDR3 sites. In a particularly preferred embodiment of the above cell, the first nucleic acid comprising the two or more PXR binding sites comprises the nucleotide sequence of SEQ ID NO:18.

In particular embodiments of the above cell, the cell further includes a second nucleic acid encoding the PXR or CAR. In further still embodiments, the cell expresses an endogenous PXR or the cell expresses an endogenous RXR. In a further still embodiment, the PXR is a human PXR or the CAR is a human CAR.

In further still embodiments, the cell is HepG2. In further still embodiments, the cell is a hepatocyte cell, in particular a hepatocyte cell selected from the group consisting of rat, mouse, and human hepatocyte cells. Preferably, the hepatocyte is a primary hepatocyte.

In further still embodiments, the reporter is secreted alkaline phosphatase (SEAP).

In a further aspect of the present invention, a method is provided for determining whether an analyte is capable of inducing expression of CYP3A4, which comprises providing a cell comprising a first nucleic acid, which includes two or more pregnane X receptor (PXR) binding sites in tandem operably linked to a heterologous promoter operably linked to a reporter gene; incubating the cell in a medium containing the analyte; and measuring expression of the reporter gene wherein an increase of the expression of the reporter gene in the presence of the analyte indicates that the analyte is capable of inducing expression of the CYP3A4. In a further embodiment, the heterologous promoter is not inducible in the absence of the two or more PXR binding sites.

In another aspect, the present invention provides a method for determining whether an analyte is capable of inducing expression of CYP3A4, which comprises providing a cell comprising a first nucleic acid, which includes two or more PXR binding sites in tandem operably linked to a heterologous promoter operably linked to a reporter gene, and a second nucleic acid encoding the PXR; incubating the cell in a medium containing the analyte; and measuring expression of the reporter gene wherein an increase of the expression of the reporter gene in the presence of the analyte indicates that the analyte is capable of inducing expression of the CYP3A4.

In a further still aspect, the present invention provides a method for determining whether an analyte is capable of inducing expression of CYP3A4, which comprises providing a cell comprising a first nucleic acid, which includes two or more PXR binding sites in tandem operably linked to a heterologous promoter, which is preferably not inducible by PXR in the absence of the PXR binding sites, operably linked to a reporter gene; incubating the cell in a medium containing the analyte; and measuring expression of the reporter gene wherein an increase of the expression of the reporter gene in the presence of the analyte indicates that the analyte is capable of inducing expression of the CYP3A4.

In a further embodiment of any one of the above methods, the PXR binding sites are selected from the group consisting of dDR3, dER6, and pER6. In further still embodiments, the dDR3 comprises the nucleotide sequence of SEQ ID NO:4, the dER6 comprises the nucleotide sequence of SEQ ID NO:7, and the pER6 comprises the nucleotide sequence of SEQ ID NO:1, or, the dDR3 comprises the nucleotide sequence of SEQ ID NO:5, the dER6 comprises the nucleotide sequence of SEQ ID NO:8, and the pER6 comprises the nucleotide sequence of SEQ ID NO:2, or, the dDR3 comprises the nucleotide sequence of SEQ ID NO:11, the dER6 comprises the nucleotide sequence of SEQ ID NO:12, and the pER6 comprises the nucleotide sequence of SEQ ID NO:10.

In a further still embodiment of the above method, the nucleic acid comprises at least one of the dER6 binding sites and at least one of the dDR3 binding sites, the nucleic acid comprises at least one of the dER6 binding sites and at least one of the pER6 binding sites, the nucleic acid comprises at least three of the dDR3 binding sites, or the nucleic acid comprises three of the dDR3 binding sites and one each of the dER6 and pER6 binding sites. In particularly preferred embodiments of the above methods, the two or more PXR binding sites in tandem of the nucleic acid comprise the nucleotide sequence of SEQ ID NO:18.

In particular embodiments of the above method, the cell further includes a second nucleic acid encoding the PXR. In further still embodiments, the cell expresses an endogenous PXR or the cell expresses an endogenous RXR. In a further still embodiment, the PXR is a human PXR.

In further still embodiments, the cell is HepG2 or a primary hepatocyte.

In further still embodiments, the reporter is secreted alkaline phosphatase (SEAP).

In a further still aspect, the present invention provides a method for determining whether an analyte is capable of inducing expression of CYP3A4, which comprises providing a primary culture of hepatocyte cells comprising a first nucleic acid, which includes two or more PXR binding sites in tandem operably linked to a heterologous promoter operably linked to a reporter gene, and a second nucleic acid encoding the PXR; incubating the culture in a medium containing the analyte; and measuring expression of the reporter gene wherein an increase of the expression of the reporter gene in the presence of the analyte indicates that the analyte is capable of inducing expression of the CYP3A4.

In a further still aspect, the present invention provides a method for determining whether an analyte is capable of inducing expression of CYP3A4, which comprises providing a primary culture of hepatocyte cells comprising a first nucleic acid, which includes two or more PXR binding sites in tandem operably linked to a heterologous promoter operably linked to a reporter gene, and a second nucleic acid encoding CAR; incubating the culture in a medium containing the analyte; and measuring expression of the reporter gene wherein an increase of the expression of the reporter gene in the presence of the analyte indicates that the analyte is capable of inducing expression of the CYP3A4.

In a further still aspect, the present invention provides a method for determining whether an analyte is capable of inducing expression of CYP3A4 via activation of a CAR, which comprises: providing a primary culture of hepatocyte cells comprising a first nucleic acid, which includes two or more PXR binding sites in tandem operably linked to a heterologous promoter operably linked to a reporter gene, and a second nucleic acid encoding the CAR; incubating the culture in a medium containing the analyte; and measuring expression of the reporter gene wherein an increase of the expression of the reporter gene in the presence of the analyte indicates that the analyte is capable of inducing expression of the CYP3A4 via activation of the CAR.

In a further still aspect, the present invention provides a method for determining whether an analyte induces expression of CYP3A4 via activation of a PXR or CAR, which comprises providing a primary culture of hepatocyte cells comprising a first nucleic acid, which includes two or more PXR binding sites in tandem operably linked to a heterologous promoter operably linked to a reporter gene, and a second nucleic acid encoding the PXR and a second primary culture of hepatocyte cells comprising the first nucleic and a third nucleic acid encoding the CAR; incubating each of the cultures in a medium containing the analyte; and measuring expression of the reporter gene wherein an increase of the expression of the reporter gene in the presence of the analyte in the first culture and not the second culture indicates that the analyte is capable of inducing expression of the CYP3A4 via activation of PXR and wherein an increase of the expression of the reporter gene in the presence of the analyte in the second culture and not the first culture indicates that the analyte is capable of inducing expression of the CYP3A4 via activation of CAR.

In further embodiments of the above methods, the hepatocyte cells are selected from the group consisting of rat, mouse, and human hepatocyte cells. In particular embodiments, it is preferred that the hepatocyte cells are rat hepatocyte cells.

In further still embodiment of any one of the above methods, the PXR binding sites are selected from the group consisting of dDR3, dER6, and pER6. In further still embodiments, the dDR3 comprises the nucleotide sequence of SEQ ID NO:4, the dER6 comprises the nucleotide sequence of SEQ ID NO:7, and the pER6 comprises the nucleotide sequence of SEQ ID NO:1, or, the dDR3 comprises the nucleotide sequence of SEQ ID NO:5, the dER6 comprises the nucleotide sequence of SEQ ID NO:8, and the pER6 comprises the nucleotide sequence of SEQ ID NO:2, or, the dDR3 comprises the nucleotide sequence of SEQ ID NO:11, the dER6 comprises the nucleotide sequence of SEQ ID NO:12, and the pER6 comprises the nucleotide sequence of SEQ ID NO:10.

In further still embodiment of the above method, the nucleic acid comprises at least one of the dER6 binding sites and at least one of the dDR3 binding sites, the nucleic acid comprises at least one of the dER6 binding sites and at least one of the pER6 binding sites, the nucleic acid comprises at least three of the dDR3 binding sites, or the nucleic acid comprises three of the dDR3 binding sites and one each of the dER6 and pER6 binding sites. In particularly preferred embodiments of the above method, the two or more PXR binding sites in tandem comprise the nucleotide sequence of SEQ ID NO:18.

In a further still aspect, the present invention provides a method for determining whether an analyte is capable of inducing expression of CYP3A4, which comprises providing a primary culture of hepatocyte cells comprising a nucleic acid, which includes two or more PXR binding sites in tandem operably linked to a heterologous promoter operably linked to a reporter gene; incubating the culture in a medium containing the analyte; and measuring expression of the reporter gene wherein an increase of the expression of the reporter gene in the presence of the analyte indicates that the analyte is capable of inducing expression of the CYP3A4.

In further still embodiment of any one of the above methods, the PXR binding sites are selected from the group consisting of dDR3, dER6, and pER6. In further still embodiments, the dDR3 comprises the nucleotide sequence of SEQ ID NO:4, the dER6 comprises the nucleotide sequence of SEQ ID NO:7, and the pER6 comprises the nucleotide sequence of SEQ ID NO:1, or, the dDR3 comprises the nucleotide sequence of SEQ ID NO:5, the dER6 comprises the nucleotide sequence of SEQ ID NO:8, and the pER6 comprises the nucleotide sequence of SEQ ID NO:2, or, the dDR3 comprises the nucleotide sequence of SEQ ID NO:11, the dER6 comprises the nucleotide sequence of SEQ ID NO:12, and the pER6 comprises the nucleotide sequence of SEQ ID NO:10.

In further still embodiment of the above method, the nucleic acid comprises at least one of the dER6 binding sites and at least one of the dDR3 binding sites, the nucleic acid comprises at least one of the dER6 binding sites and at least one of the pER6 binding sites, the nucleic acid comprises at least three of the dDR3 binding sites, or the nucleic acid comprises three of the dDR3 binding sites and one each of the dER6 and pER6 binding sites. In particularly preferred embodiments of the above method, the two or more PXR binding sites in tandem comprise the nucleotide sequence of SEQ ID NO:18.

The present invention further provides a kit, which comprises a first container, which includes a first nucleic acid, which includes two or more PXR binding sites in tandem operably linked to a heterologous promoter operably linked to a reporter gene. Preferably, the heterologous promoter is not inducible by PXR in the absence of the PXR binding sites. Optionally, a second container is provided, which includes a second nucleic acid, which encodes PXR and is operably linked to a heterologous promoter. In further aspects of the kit, the first container comprises a reporter gene expression cassette that includes a first nucleic acid comprising in tandem two or more PXR binding sites, which is operably linked to a second nucleic acid bearing a heterologous promoter that is not inducible by PXR in the absence of the nucleic acid comprising the PXR binding sites, operably linked to a third nucleic acid encoding a reporter gene.

In further aspects of the kit, the kit further including reagents for measuring expression of the reporter gene. In further still aspects, the kit further includes reagents for transfecting the nucleic acids into a cell. In further still aspects, the kit further includes cells.

In a further embodiment of the kit, the PXR binding sites are selected from the group consisting of dDR3, dER6, and pER6. In further still embodiments, the dDR3 comprises the nucleotide sequence of SEQ ID NO:4, the dER6 comprises the nucleotide sequence of SEQ ID NO:7, and the pER6 comprises the nucleotide sequence of SEQ ID NO:1, or, the dDR3 comprises the nucleotide sequence of SEQ ID NO:5, the dER6 comprises the nucleotide sequence of SEQ ID NO:8, and the pER6 comprises the nucleotide sequence of SEQ ID NO:2, or, the dDR3 comprises the nucleotide sequence of SEQ ID NO:1, the dER6 comprises the nucleotide sequence of SEQ ID NO:12, and the pER6 comprises the nucleotide sequence of SEQ ID NO:10.

In a further still embodiment of the above kit, the first nucleic acid comprises at least one of the dER6 binding sites and at least one of the dDR3 binding sites, the nucleic acid comprises at least one of the dER6 binding sites and at least one of the pER6 binding sites, the nucleic acid comprises at least three of the dDR3 binding sites, or the nucleic acid comprises three of the dDR3 binding sites and one each of the dER6 and pER6 binding sites. In further still embodiments, the nucleic acid comprises at least three dDR3 sites. In particularly preferred embodiments of the above method, the two or more PXR binding sites in tandem comprise the nucleotide sequence of SEQ ID NO:18.

In particular embodiments of the kit, the reporter is secreted embryonic alkaline phosphatase (SEAP).

Preferably, in the above methods and kit, the first nucleic acid is the reporter gene expression cassette as described above. Preferably, in particular embodiments of the above methods described herein, the second nucleic acid is a gene expression cassette comprising a first nucleic acid encoding PXR or CAR operably linked to a second nucleic acid bearing a heterologous promoter, preferably a promoter that is not inducible by PXR or CAR.

### BRIEF DESCRIPTION OF THE DRAWINGS

FIG. 1 shows a cartoon illustrating a prior art CYP3A4 induction assay using HepG2 cells transfected with human PXR and a plasmid consisting of the prior art ΔCYP3A4 promoter operably linked to the SEAP reporter gene.

FIG. 2A is a schematic diagram of the ΔCYP3A4 promoter of the prior art operably linked to the SEAP reporter gene. Shown are the locations for the human PXR or CAR binding sites dDR3, dER6 and pER6.

FIG. 2B is a schematic diagram of the strategy for generating composite promoters responsive to human PXR or CAR induction.

FIG. 3A shows the nucleotide sequence for a double-stranded oligomer comprising the pER6 forward strand (SEQ ID NO:2) and complementary strand (SEQ ID NO:3). The base number refers to the human CYP3A promoter region.

FIG. 3B shows the nucleotide sequence for the double-stranded oligomer comprising the dER6 forward strand (SEQ ID NO:5) and complementary strand (SEQ ID NO:6). The base number refers to the human CYP3A promoter region.

FIG. 3C shows the nucleotide sequence for the double-stranded oligomer comprising the dDR6 forward strand (SEQ ID NO:8) and complementary strand (SEQ ID NO:9). The base number refers to the human CYP3A promoter region.

FIG. 4A shows a schematic diagram of clone 102-SEAP (plasmid pVIj-SEAP-polyEcoRV-#102) comprising the SEAP reporter gene expression cassette which comprises the SEAP reporter gene operably linked the composite promoter comprising the clone 102 enhancer and CMV minimal promoter.

FIG. 4B shows a schematic diagram of the clone 102-SEAP reporter gene cassette.

FIG. 4C shows the nucleotide sequence of the reporter gene cassette shown in FIG. 4B (SEQ ID NO:22). The clone 102 enhancer is in capital letters, the CMV minimal promoter is underlined, and the SEAP reporter gene is in italics.

FIG. 5A shows a schematic diagram of plasmid pVIj-SEAP-polyEcoRV.

FIGS. 5B and 5C provide the nucleotide sequence of pVIj-SEAP-polyEcoRV (SEQ ID NO:21). The nucleotide sequence corresponding to the EcoRV restriction enzyme site is underlined.

FIG. 6 shows a schematic diagram showing the location and distribution of the dDR3, dER6, and pER6 enhancer elements in the composite enhancer of several of the composite promoters of the present invention. The orientation of the element is indicated by the arrowhead. For the enhancer elements comprising the each of the clones, arrowheads pointing to the right indicate that the enhancer element is in the native orientation whereas arrowheads pointing to the left are in the non-native orientation. The dDR3 is represented by the white arrow, the dER6 is represented by the black arrow, and the prER6 is represented by the cross-hatched arrow.

FIG. 7 shows a schematic diagram of plasmid pZDCVS-ΔCYP3A4/SEAP.

FIG. 8A shows a schematic diagram of plasmid pSG5-dATG-hPXR.

FIG. 8B shows a schematic diagram of plasmid pCR3.1-hCAR.

FIG. 9 shows the results of an assay testing clones 3, 21, 26, 33, 58, 61, 70, 71, and 102 from the composite promoter library co-transfected into HepG2 cells in the presence of a nuclear receptor (NR) donor plasmid encoding human PXR for inducibility of SEAP transcription in the presence and absence of 10 mM Rifampicin. Reporter plasmid ΔCYP3A4/SEAP was included as a positive control. Negative controls omitted the NR donor plasmid. Expression of SEAP is reported as SEAP arbitrary units. Numbers on top of the bars represent the fold induction of transcription from the promoter in the presence or absence of rifampicin.

FIG. 10 shows the results of an assay testing inducibility of the composite promoter of clone 102-SEAP in the presence and absence of various inducers in the presence of NR donor DNA encoding human PXR. Negative controls omitted the NR donor plasmid. Expression of SEAP is reported as arbitrary SEAP unit. Numbers on top of the bars represent the fold induction of transcription from the clone 102-SEAP composite promoter in the presence or absence of the indicated compounds.

FIG. 11 shows the results of an assay testing inducibility of the composite promoter of library clone 102-SEAP in the presence and absence of various inducers in the presence of NR donor DNA encoding human PXR. Negative controls omitted the NR donor plasmid. Expression of SEAP is reported as percentage of the induction of transcription by Rifampicin, which was set at 100%. Numbers on top of the bars represent the fold induction of transcription from the clone 102-SEAP composite promoter in the presence or absence of the indicated compounds relative to induction by Rifampicin. The results are shown in comparison to the inducibility of the ΔCYP3A4 promoter in ΔCYP3A4/SEAP.

FIG. 12 shows the results of an assay testing inducibility of the composite promoter of clone 102-SEAP in the presence and absence of various inducers in the presence of NR donor DNA encoding human CAR. Negative controls omitted the NR donor plasmid. Expression of SEAP is reported as arbitrary SEAP unit. Numbers on top of the bars represent the fold induction of transcription from the clone 102-SEAP composite promoter in the presence or absence of the indicated compounds. ΔCYP3A4/SEAP was included as a positive control.

FIG. 13 shows the results of an assay testing inducibility of the composite promoter of clone 102-SEAP in the presence and absence of various inducers in the presence of NR donor DNA encoding human CAR. Negative controls omitted the NR donor plasmid. Expression of SEAP is reported as percentage of the induction of transcription by Rifampicin, which was set at 100%. Numbers on top of the bars represent the fold induction of transcription from the clone 102-SEAP composite promoter in the presence or absence of the indicated compounds relative to induction by Rifampicin. The results are shown in comparison to the inducibility of the ΔCYP3A4 promoter in ΔCYP3A4/SEAP.

FIG. 14 shows that primary rat hepatocytes do not contain an endogenous PXR or CAR activity that activated transcription from clone 102-SEAP when transfected into the hepatocytes alone. The Figure shows that when clone 102-SEAP DNA was cotransfected into the hepatocytes with a gene cassette encoding CAR, only CAR activators were able to induce transcription from clone 102-SEAP. The Figure also shows that when clone 102-SEAP DNA was cotransfected into the hepatocytes with a gene cassette encoding PXR, only PXR activators were able to induce transcription from clone 102-SEAP. Results are expressed as SEAP arbitrary units.

FIG. 15 shows that primary human hepatocytes transfected with clone 102-SEAP can detect inducers of CYP3A4 via PXR or CAR. Results are expressed as SEAP arbitrary units.

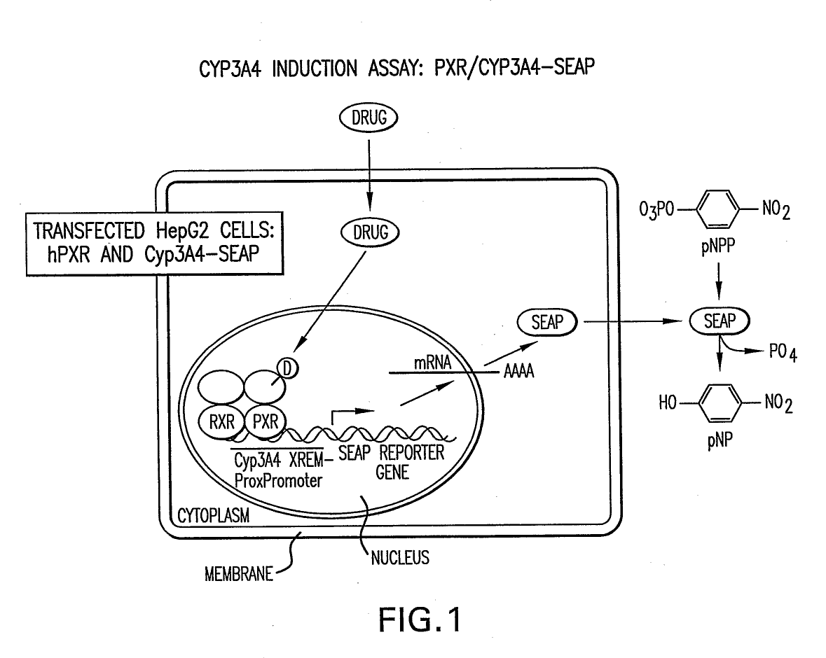

### DETAILED DESCRIPTION OF THE INVENTION

The present invention provides an assay for screening analytes (molecules, compounds, drug candidates, or the like) for ability to mediate transcriptional activation (or expression) of various members of the cytochrome P-450 (P450) superfamily of hemoproteins, in particular, the CYP3A4 isoform. The assay comprises providing a recombinant cell, which expresses the pregnane X receptor (PXR) or constitutive androstane receptor (CAR), and which comprises therein a reporter gene operably linked to a novel composite or synthetic PXR- or CAR-inducible promoter comprising multiple copies of at least one of the three enhancer elements of the CYP3A4 promoter in a tandem array operably linked to a minimal promoter, incubating the recombinant cell in the presence of an analyte, and measuring expression of the reporter gene. An analyte, which is a mediator of transcriptional activity from the CYP3A4 promoter, exerts its effect in the assay by binding to the PXR or CAR and activating the PXR or CAR. The activated PXR or CAR then induces expression of the reporter gene via the PXR- or CAR-inducible composite promoter. An analyte that is not a mediator of transcriptional activity of PXR or CAR either does not bind and activate the PXR or CAR or binds the PXR or CAR but doe not activate the PXR or CAR. In either case, there is no transcription from the PXR- or CAR-inducible composite promoter.

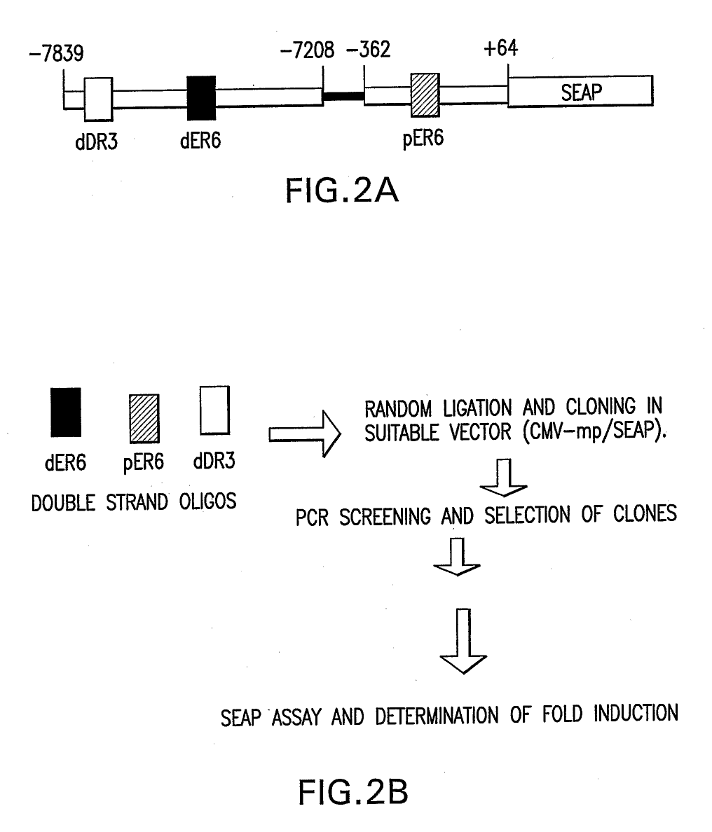

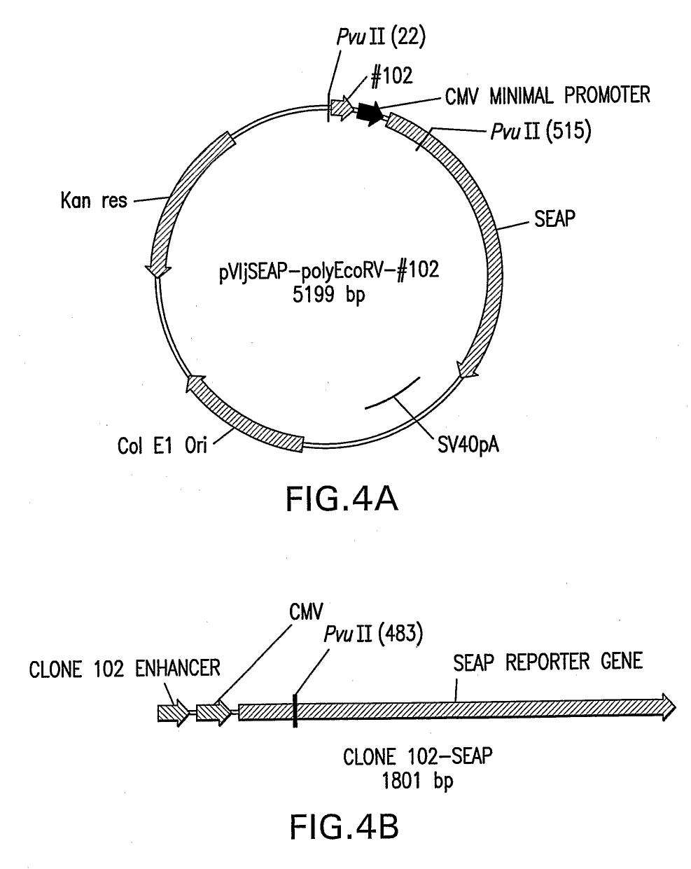

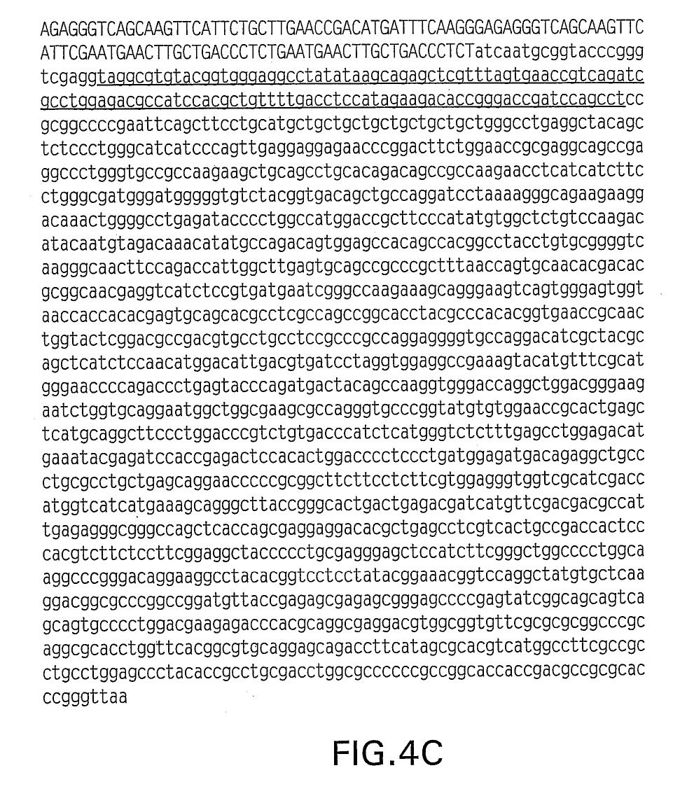

The novel composite promoter of the present invention provides a higher fold induction in response to a transcriptional activation than the native CYP3A4 promoter. For example, the CYP3A4 transcription inducer rifampicin causes a greater fold induction of a reporter gene operably linked to the composite promoter of the present invention than a reporter gene operably linked to the native CYP3A4 promoter or variants thereof such as the prior art deletion CYP3A4 (ΔCYP3A4) promoter consisting of the CYP3A4 promoter region from about −7839 to −6000 bp linked to the region from about −362 to 53 bp which is then operably linked to a reporter gene (See FIG. 2A or 4; Goodwin et al., Mol. Pharmacol. 56: 1329-1339 (1999); Drocourt et al., Drug. Metab. Disp. 29: 1325-1331 (2001); Example 2). The higher sensitivity of the composite promoter of the present invention over the native CYP3A4 promoter or ΔCYP3A4 promoter provides a more sensitive assay for screening compounds and drug candidates for CYP3A4 inducibility. The present invention further provides a method for making composite promoters which are responsive to PXR or CAR binding or activation.

In general, a promoter consists of two elements, (1) a minimal promoter element which includes the core promoter and the 5′ untranslated region (UTR) of the transcription unit and (2) one or more enhancer elements consisting of binding sites for transcription activators. A core promoter is a short nucleotide sequence that mediates initiation of transcription. In general, a core promoter contains a TATA box and a G-C rich region associated with a CAAT box. These elements act to bind RNA polymerase II to the promoter and assist the polymerase in locating the RNA initiation site. Some promoters do not have a TATA box or CAAT box but instead contain an initiator element that encompasses the transcription initiation site. The 5′ UTR plays a role in enhancing the stability of the RNA transcript. Longer 5′ UTRs usually contain an intron with regulatory sequences that modulate gene expression at the transcriptional or translational level. Enhancer elements are nucleotide sequences that enhance the amount of RNA transcribed from a particular promoter. Enhancer elements can be immediately upstream of the core promoter (proximal enhancers) or several thousand base pairs upstream or downstream from the promoter (distal enhancers). Enhancer elements contain therein one or more short nucleotide sequences called response elements. The response elements bind transcription factors which enhance the formation of the RNA transcription initiation complex. Native promoters (or minimal promoters) consist of a single nucleotide fragment from the 5′ end of a transcription unit. In general, native promoters contain a core promoter and 5′UTR and depending on the length of the nucleotide fragment, some native promoters can further contain one or more of its enhancer elements, and optionally, an intron. In general, a composite promoter consists of a core promoter, one or more enhancer elements, and 5′ UTR of which at least two are of a different origins or combines a distal enhancer element with a minimal promoter of the same origin.

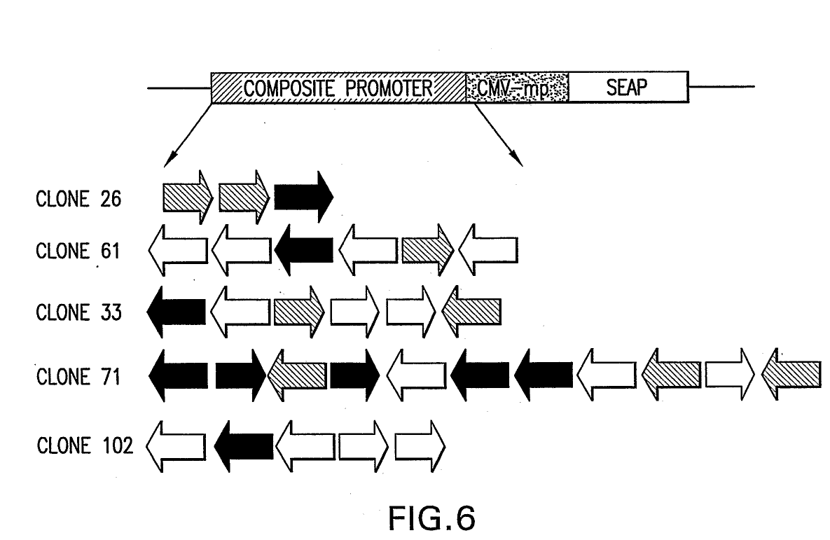

The composite PXR- or CAR-inducible promoter of the present invention comprises a minimal or core promoter operably linked in cis with a composite enhancer comprising at least two enhancer elements consisting of PXR or CAR binding sites, which enable transcription from the promoter to be regulated or induced by PXR or CAR. Each enhancer element (or binding site) comprises an oligomer comprising one PXR or CAR binding site, which in the composite enhancer are, in general, in a tandem arrangement. The relationship of each enhancer element oligomer to its neighboring enhancer element oligomer can be head-to-tail, head-to-head, or tail-to-tail (See FIG. 6 for examples of arrangements of the oligomers). In some embodiments, one or more of the enhancer element oligomers are not in the tandem arrangement but are located in position at a distance from the other enhancer element oligomers of the composite enhancer. The enhancer element oligomers of the composite enhancer can be located upstream or downstream from the minimal or core promoter. The enhancer element oligomers of the composite enhancer can be located adjacent to the minimal or core promoter or located at a distance away from the minimal or core promoter. For example, the composite enhancer can be located at the distal end of the minimal or core promoter or located up to about 13 kb upstream from the distal end (5′) of the minimal or core promoter. Alternatively, the composite enhancer can be located at the proximal end (3′) of the minimal or core promoter or located up to about 13 kb downstream from the proximal end of the minimal or core promoter. For example, the PXR- or CAR-inducible reporter can comprise a minimal or core promoter at the proximal end (5′ end) of the reporter gene and the enhancer elements at the distal end (3′) of the reporter. In further embodiments, the composite enhancer consists of a enhancer element oligomers located at different positions in and around the minimal promoter such that one part of the composite enhancer is located upstream of the minimal promoter and another part of the composite enhancer is located at another position, for example, downstream of the minimal promoter. As used herein, operably linked means that the minimal promoter is under the control (part or fall) of the composite enhancer. It does not require the enhancer element or composite enhancer to be immediately adjacent to the minimal promoter. The minimal or core promoter is preferably not of CYP3A4 origin and is preferably, is not inducible by PXR or CAR in the absence of the enhancer elements.

The native CYP3A4 promoter has three enhancer elements or PXR/CAR binding sites that enable PXR or CAR induction of the CYP3A4 promoter. These enhancer elements are the proximal enhancer element, pER6 (prPXRE or ER6), which consists of an everted repeat located in the region of the CYP3A4 promoter from nucleotide −176 to −146, and the first and second distal enhancer elements, dDR3 (direct repeat or dNR1 or DR3) and dER6 (everted repeat or dNR2 or ER6), respectively, which are located in the region of the CYP3A4 promoter from nucleotide −7839 to −7208 (the XREM region). The entire CYP3A4 promoter spans about 7.8 kb. In contrast; the ΔCYP3A4 promoter is about 2254 bp. FIG. 2A shows the location of the enhancer elements in the ΔCYP3A4 promoter.

The proximal enhancer element or PXR/CAR binding site, pER6, consists of the hexamer, 5′-TGAMCT-3′, separated by six nucleotides from its everted repeat, 5′-AGKTCA-3′, wherein M is A or C and K is G or T. Thus, the pER6 comprises the forward strand nucleotide sequence 5′-TGAMCT-N₆-AGKTCA-3′ (SEQ ID NO:1) wherein M is A or C, K is G or T, and N is any nucleotide, and its complementary strand. The pER6 enhancer element of the CYP3A4 promoter is from the region of the CYP3A4 promoter encompassed by nucleotides −176 to −146, which has the forward strand nucleotide sequence 5′-TAGAATATGAACTCAAAGGAGGTCAGTGAGT-3′ (SEQ ID NO:2). The everted repeats are underlined.

The first distal enhancer element or PXR/CAR binding site, dDR3, consists of a pair of direct repeats of the hexamer 5′-TGAMCY-3′ wherein M is A or C and Y is T or C, each hexamer separated by three nucleotides. Thus, the dDR3 comprises the forward strand nucleotide sequence 5′-TGAMCY-N₃-TGAMCY-3′ (SEQ ID NO:4) wherein M is A or C and Y is T or C, and its complementary strand. The dDR3 enhancer element of the CYP3A4 promoter is encompassed by nucleotides −7736 to −7716 which has the forward strand nucleotide sequence 5′-GAATGAACTTGCTGACCCTCT-3′ (SEQ ID NO:5). The direct repeats are underlined.

The second distal enhancer element or PXR/CAR binding site, dER6, consists of the hexamer 5′-TGAAMY-3′ separated by six nucleotides from its everted repeat 5′-KRTTCA-3′ wherein M is A or C, Y is T or C, K is G or T, and R is G or A. Thus, the dER6 comprises the forward strand nucleotide sequence 5′-TGAAMY-N₆-KRTTCA-3′ (SEQ ID NO:7) wherein M is A or C, K is G or T, R is G or A, and N is any nucleotide, and its complementary strand. The dER6 enhancer element of the CYP3A4 promoter is from the region of the CYP3A4 promoter encompassed by nucleotides −7693 to −7668, which has the forward strand nucleotide sequence 5′-CCCTTGAAATCATGTCGGTTCAAGCA-3′ (SEQ ID NO:8). The everted repeats are underlined.

In the composite promoter of the present invention, the pER6 comprises an oligomer comprising the nucleotide sequence 5′-TGAMCT-N₆-AGKTCA-3′ (SEQ ID NO:1), the dDR3 comprises an oligomer comprising the nucleotide sequence 5′-TGAMCY-N₃-TGAMCY-3′ (SEQ ID NO:4), and the dER6 comprises an oligomer comprising the nucleotide sequence 5′-TGAAMY-N₆-KRTTCA-3′ (SEQ ID NO:7), wherein M is A or C, K is G or T, R is G or A, and Y is T or C, and N is any nucleotide. Examples of pER6 enhancer elements embraced by the composite promoter of the present invention include 5′-TGAACTCAAAGGAGGTCA-3′ (SEQ ID NO:10) and 5′-TAGAATATGAACTCAAAGGAGGTCAGTGAGT-3′ (SEQ ID NO.2). Examples of the dDR enhancer elements include 5′-TGAACTTGCTGACCC-3′ (SEQ ID NO:11) and 5′-GAATGAACTTGCTGACCCTCT-3′ (SEQ ID NO.5). Examples of dER6 enhancer elements include 5′-TGAAATCATGTCGGTTCA-3′ (SEQ ID NO:12) and 5′-CCCTTGAAATCATGTCGGTTCAAGCA-3′ (SEQ ID NO:8).

The composite promoter of the present invention comprises in tandem two or more oligomers, each oligomer comprising a PXR or CAR binding site, operably linked to a heterologous promoter, preferably a heterologous minimal promoter. Thus, the composite promoter comprises a composite enhancer, operably linked to a heterologous promoter. In a preferred aspect, the composite enhancer comprises one or more copies of each of the dDR3 and dER6 enhancer elements or PXR/CAR binding sites and optionally, one or more copies of the pER6 enhancer elements or PXR/CAR binding sites or one or more copies of each of the pER6 and dER6 enhancer elements and optionally, one or more copies of the dDR3 enhancer elements arranged in various configurations, orientations, and copy numbers. Preferably, the composite enhancer comprises at least two different enhancer elements or PXR/CAR binding sites (for example, at least one each of dDR3 and dER6). Preferably, in particular aspects, the composite enhancer comprises at least three dDR3 enhancer elements. By way of example, FIG. 6 illustrates several arrangements of enhancer elements operably linked to the cytomegalovirus (CMV) minimal promoter in several of the composite promoters of the present invention. As shown in FIG. 6, the composite enhancer of the present invention comprises the PXR/CAR binding sites in a tandem arrangement wherein each PXR/CAR binding site is adjacent to another PXR/CAR binding site. FIG. 6 also shows that the orientation of the PXR/CAR binding sites in the composite enhancer include any combination of head-to-head, tail-to-head, or tail-to-tail orientations. For example, the composite promoter of clone 26-SEAP of Example 1 comprises a composite enhancer consisting of two copies of pER6 and one copy of dER6; all three enhancer elements in the native orientation, operably linked to a CMV minimal promoter. Clone 102-SEAP of Example 1 comprises a composite enhancer consisting of one copy of dER6 and four copies of dDR3 with only one copy of the dDR3 elements is in the native orientation, operably linked to the CMV promoter.

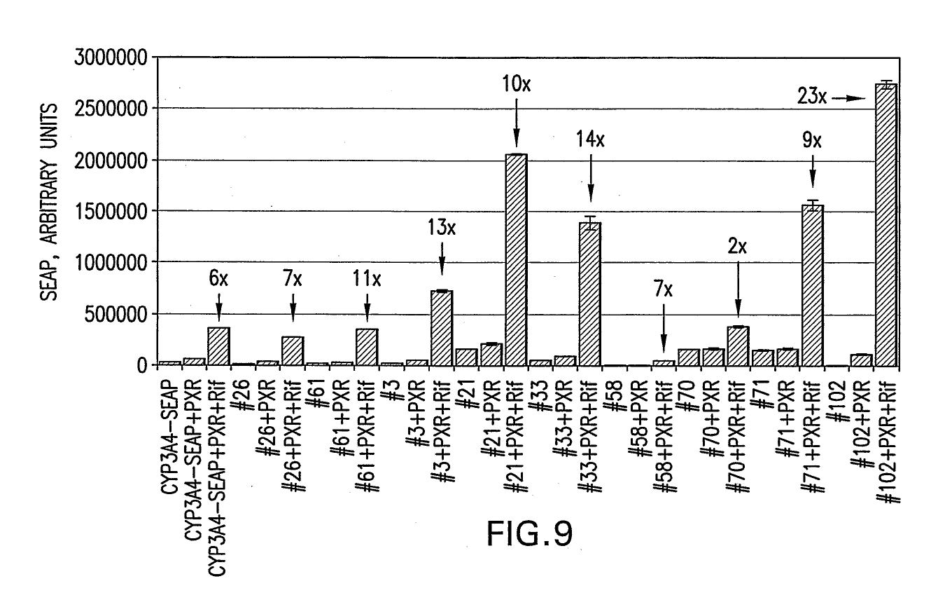

It was observed that several of the composite promoters shown in FIG. 6 produced a particularly strong signal in response to the CYP3A4 activator Rifampicin compared to the response of the prior art ΔCYP3A4 promoter. As shown in FIG. 9, clone 102-SEAP gave a signal that was 23-fold over background, clone 33-SEAP gave a signal that was 14-fold over background, and clone 61-SEAP gave a signal that was 11-fold over background. In contrast, the ΔCYP3A4 promoter of the prior art gave a signal that was only 6-fold over background. The unexpected increase in signal strength of the above composite promoters enabled development of assays for identifying activators of CYP3A4 which are more sensitive and robust over prior art assays. The 23-fold increase in signal strength of the clone 102-SEAP over background further enabled development of an assay that can be used to identify analytes that are activators of CAR from analytes that are activators of PXR.

As shown by the Examples herein, the composite promoter can comprise a composite enhancer, which comprises one or more copies of pER6 and dER6 enhancer elements and preferably further including one or more copies of the dDR3 enhancer element in any order, operably linked to a cytomegalovirus immediate early 1 (CMV) minimal promoter. The nucleotide sequence of the CMV minimal promoter comprises 5′-TAGGCGTGTA CGGTGGGAGG CCTATATAAG CAGAGCTCGT TTAGTGAACC GTCAGATCGC CTGGAGACGC CATCCACGCT GTTTTGACCT CCATAGAAGA CACCGGGACC GATCCAGCCT-3′ (SEQ ID NO:13). The present invention is not limited to the CMV minimal promoter but can include an alternative minimal promoter. Examples of other minimal promoters or promoters of which the minimal part thereof can be obtained include, but are not limited to, the native promoter for β-Actin (β-Act), alpha-fetoprotein (AFP), immunoglobulin beta (B29), monocyte receptor for bacterial LPS (CD14), leukosialin, sialophorin (CD43), leukocyte common antigen (LCA) (CD45), homolog of macrosialin (CD68), carcinoembryonic antigen (CEA), c-erbB2/neu oncogene (c-erbB2), cyclo-oxygenase 2 isoform (prostaglandin-endoperoxide synthase 2) (COX-2), desmán, elongation factor 1 (EF1), E2F transcription factor 1 (E2F-1), early growth response 1 (EGR1), eukaryotic initiation factor 4A1 (eIF4A1), elastase-1, endoglin (ENG), ferritin heavy chain (FerH), ferritin light chain (FerL), fibronectin (FN), VEGF-receptor 1 (Flt-1), glyceraldehyde 3-phosphate dehydrogenase (GAPDH), glial fibrillary acidic protein (GFAP), glucose-regulated protein 78 (GRP78), glucose-regulated protein 94 (GRP94), heat shock protein 70 (HSP70), herpes simplex virus thymidine kinase (hsvTK), intercellular adhesion molecule 2 (CD102) (ICAM-2), interferon beta (IFNB), beta-kinesin (β-Kin), L-plastin (lymphocyte cytosolic protein 1) (LP), myoglobin (Mb), mucin-like glycoprotein (breast carcinoma-associated antigen, DF3) (MUC1), osteocalcin-2 (OG-2), phosphoglycerate kinase (PGK-1), prostate specific antigen (PSA), surfactant protein B (SP-B), synapsin I (SYN1), tyrosinase related protein (TRP1), tyrosinase (Tyr), and ubiquitin B (Ubi B).

The composite promoter comprising a composite enhancer, which comprises two or more oligomers comprising enhancer elements or PXR/CAR binding sites, operably linked to a minimal promoter is further operably linked to a reporter gene that encodes an assayable product to provide a reporter gene expression cassette. Preferably, the composite enhancer comprises at least two different enhancer elements or PXR/CAR binding sites (for example, at least one each of pER6 and dER6), or more preferably, three different enhancer elements or PXR/CAR binding sites (for example, at least one each of prER6, dER6, and dDR3). It is further preferable that the reporter gene expression cassette comprises a 3′ UTR with a polyadenylation site and a transcription termination site downstream of the reporter gene. In a particularly preferred embodiment, the promoter of the reporter gene expression cassette comprises the composite promoter of clone 102-SEAP or the nucleotide sequence of SEQ ID NO:18 operably linked to a heterologous promoter such as the CMV minimal promoter.

In general, the reporter gene expression cassette is included as a component of a vector such as a plasmid, cosmid, phagemid, virus, bacteriophage, transposon, artificial chromosome, or other vector that can be transfected into eukaryote cells. In general, it is preferable that for vectors which are plasmids, the vector comprise an origin of replication such as the SV40 origin of replication, which enables the vector to be propagated in eukaryote cells. It is further preferable that the vector include a means for selecting or identifying recombinant cells which contain the vector. For example, the vector can contain a gene that confers neomycin or kanamycin resistance to transfected cells that contain the vector or a gene such as green fluorescent protein or luciferase that enables the transfected cells to separated from non-transfected cells using a cell sorter or the like. As an example, the pVIj-SEAP-polyEcoRV vector comprises the secreted embryonic alkaline phosphatase (SEAP) reporter gene operably linked to the CMV minimal promoter. As shown in the examples, the vector was used to construct several vectors, each comprising a tandem array of PXR/CAR binding sites operably linked to the CMV minimal promoter to provide a composite promoter operably linked to a SEAP reporter gene. Several of the above vectors have been used to determine whether an analyte can induce CYP3A4 expression via PXR or CAR activation.

The present invention further provides recombinant host cells, which have been transformed or transfected with a vector comprising any one of the aforementioned nucleic acid molecules, particularly recombinant host cells, which have been transformed or transfected with at least a vector comprising a reporter gene expression cassette reporter gene. For example, the host cells can be transformed or transfected with a reporter gene expression cassette comprising the composite promoter of clone 102-SEAP or the nucleotide sequence of SEQ ID NO:18 operably linked to a heterologous promoter operably linked to a reporter gene. In particular embodiments, it is desirable that the composite promoter be operably linked to the reporter gene encoding SEAP. In particular embodiments, the host cells are further transfected or transformed with a vector comprising a gene expression cassette encoding PXR or CAR or both. Recombinant host cells include bacteria such as E. coli, fungal cells such as yeast, plant cells, mammalian cells including, but not limited to, cell lines of bovine, porcine, non-human primate, human, or rodent origin; and insect cells including, but not limited to, Drosophila and silkworm-derived cell lines. For instance, one insect expression system utilizes Spodoptera frugiperda (Sf21) insect cells (Invitrogen) in tandem with a baculovirus expression vector (pAcG2T, Pharmingen, San Diego, Calif.).

Preferably, the recombinant cell is a mammalian cell, preferably, a rat, mouse, primate, or human cell. The recombinant cell can be made from either primary cells or an immortal cell line. In particular embodiments, it is preferable that the recombinant cell be derived from a cell which produces RXR endogenously. As disclosed in the Examples herein, the reporter gene expression cassette was transfected into HepG2 cells, an immortal cell line derived from a human hepatocellular carcinoma, or in primary rat or human hepatocyte cells to produce novel recombinant cells for use in the method of the present invention. HepG2 cells normally produce high levels of endogenous RXR, a particularly desirable phenotype. HepG2 cells are disclosed in U.S. Pat. No. 4,393,133 to Knowles. Sources for HepG2 cells include the Istituto Zooprofilattico Sperimentale (accession number IZSBS BS TCL79; IZSBS, Via A. Bianchi 7, Brescia, 25100 Italy); the American Type Culture Collection (accession numbers HB8065 and CRL-11997; ATCC, 10801 University Boulevard, Manassas, Va.; 20110); and, the German Collection of Microorganisms and Cell Cultures (accession number ACC 180; DSMZ, Mascheroder Weg 1b, Braunschweig, D-38124 Germany). Other cell lines include, but are not limited to, H-4IIE cells (ATCC CRL-1548), HeLa (ATCC CCL-2), Hep3b (ATCC HB8064), WiDr (ATCC CCL-218), HCT116 (ATCC CCL-247), MCF-7 (ATCC HTB-22), and 293 (ATCC CRL-1573).

In particular, the present invention provides recombinant cells comprising a reporter gene expression cassette comprising a composite enhancer, which comprises two or more enhancer elements or PXR/CAR binding sites, operably linked to a heterologous promoter that is not inducible by PXR in the absence of the enhancer elements or PXR binding sites, to make a composite promoter that is operably linked to a reporter gene. Preferably, the heterologous promoter is a minimal promoter such as the CMV minimal promoter. Preferably, the composite enhancer comprises at least two different enhancer elements or PXR/CAR binding sites (for example, at least one each of dDR3 and dER6). Preferably, in particular aspects, the composite enhancer comprises at least three dDR3 enhancer elements (for example, the composite enhancers shown in FIG. 6 each contain at least three copies of dDR3). In particular preferred embodiments, the composite promoter comprises the composite promoter of clone 102-SEAP or the nucleotide sequence of SEQ ID NO:18 operably linked to a heterologous promoter. The reporter gene expression cassette comprising the PXR- or CAR-inducible reporter can be transiently transfected into the recombinant cell or stably integrated into the genome of the recombinant cell.

PXR or CAR can be endogenously or ectopically expressed in the recombinant cell. Preferably, the recombinant cell further comprises a second nucleic acid encoding the PXR or CAR operably linked to a constitutive promoter or to an inducible promoter for ectopic expression of the PXR or CAR. The nucleotide sequence for the human PXR has been disclosed in Willson and Kliewer et al., Cell 92: 73-82 (1998), WO 9935246 (and U.S. Pat. No. 6,756,491) to Evans and Blumberg, WO 9948915 to Kliewer et al., and WO 9919354 to Berkenstam and Dahlberg and is GenBank accession number AF061056. PXR from mouse; rat, and rabbit have been cloned and sequenced (Klieweer et al., Cell 92: 73-82 (1998); Jones et al., Molec. Endocrinol. 14: 27-39 (2000); Zhang, Arch. Biochem. Biophys. 368: 14-22 (1999)). Examples of other non-human PXRs are disclosed in WO 02094865 to Kliewer et al. The nucleotide sequence for the human CAR has been disclosed in WO 9317041 to Moore (and U.S. Pat. Nos. 5,686,574, 5,710,017, and 5,756,448) and Baes and U.S. Pat. No. 6,579,686 to Collins et al. Preferably, the promoter operably linked to the PXR or CAR is a constitutive promoter which can be a naturally occurring promoter or a composite promoter. The second nucleic acid can be transiently transfected into the recombinant cell or stably integrated into the genome of the recombinant cell. The recombinant cell preferably expresses the retinoid X receptor (RXR) as well. Expression of the RXR can be endogenous or can be ectopic by providing a third nucleic acid encoding the RXR operably linked to a constitutive or inducible promoter which is transiently transfected into the recombinant cell or stably integrated into the genome of the recombinant cell. Preferably, the RXR expression is endogenous. When the first, second, and optionally, the third, nucleic acids are stably integrated into the cell, a cell line is produced which simplifies the method for identifying analytes which affect expression of CYP3A4 because the method does not need to be preceded by a transfection step.

Methods for producing transiently or stably transfected eukaryote cells are well known in the art and can be found for example in Sambrook et al., Molecular Cloning: A Laboratory Manual 2nd Edition; Cold Spring Harbor Laboratory Press, Cold Spring Harbor, N.Y., (1989) or Sambrook and Russell, Molecular Cloning: A Laboratory Manual, 3rd Edition. Cold Spring Harbor Laboratory Press, Plainview, N.Y. (2001)).

For example, to make transiently transfected cells that comprise the gene expression reporter cassette and a nucleic acid encoding PXR or CAR, the cells are plated in tissue culture plates and transfected with a first nucleic acid comprising the reporter gene expression cassette and a second nucleic acid encoding PXR or CAR. Currently, it is preferable that the PXR or CAR be of human or primate origin. The nucleic acids can be provided as a component of any one of the aforementioned vectors, for example, as a component of a plasmid. The nucleic acid encoding the gene expression reporter cassette and the nucleic acid encoding the PXR or CAR can be provided in separate vectors or as components of the same vector. In some aspects, the transfection includes a third nucleic acid encoding RXR. The third nucleic acid can be a component of the same vector containing the first and second nucleic acids. After incubating the cells for a time sufficient for the nucleic acid to be taken up by the cells, the transfection medium is removed and replaced with medium containing an analyte to be tested for CYP3A4 activity. After sufficient time for the transfected nucleic acid to be expressed, usually about 48 hours, expression of the reporter gene is assayed.

For example, to make stably transfected cells, the cells are plated in tissue culture plates and transfected with a first nucleic acid comprising the reporter gene expression cassette and a second nucleic acid encoding PXR or CAR. Currently, it is preferable that the PXR or CAR be of human or primate origin. The nucleic acids further include a gene which encodes a means for selecting cells which contain the nucleic acids. Genes which are useful for selecting recombinant cells include, but are not limited to, the neomycin gene which confers resistance to G418 or kanamycin, the pac gene which confers resistance to puromycin, the hygromycin resistance gene which confers resistance to hygromycin B, and the xanthine guanine phosphoribosyltransferase which confers resistance to mycophenolic acid. The nucleic acids can be provided as a component of any one of the aforementioned vectors, for example, as a component of a plasmid. The nucleic acid encoding the gene expression reporter cassette and the nucleic acid encoding the PXR or CAR can be provided in separate vectors or as components of the same vector. In some aspects, the transfection includes a third nucleic acid encoding RXR. The third nucleic acid can be a component of the same vector containing the first and second nucleic acids. After incubating the cells for a time sufficient for the nucleic acid to be taken up by the cells, the transfection medium is removed and replaced with medium containing a means for selecting transfected cells from non-transfected cells. Examples of selection means include, but are not limited to, G418, kanamycin, hygromycin B, mycophenolic acid, and puromycin. To select recombinant cells using G148, the above vector further includes a gene which encodes neomycin. After several passages of the cells in medium containing the selection means, the non-transfected cells die off and the recombinant cells remain. The recombinant cells can be cultivated to provide stocks of recombinant cells which can be used as described below for determining whether an analyte is an activator of CYP3A4 expression.

In general, the method of the present invention for identifying analytes which are activators of CYP3A4 expression comprises the following steps. A recombinant cell prepared as disclosed herein is provided which includes therein a nucleic acid which comprises a gene expression cassette, which includes a reporter gene operably linked to a composite promoter as disclosed herein. In a preferred embodiment, the composite promoter comprises the composite promoter of clone 102-SEAP or a composite promoter comprising the nucleotide sequence of SEQ ID NO:18 operably linked to a heterologous promoter. The gene expression cassette can be provided on a vector that can replicate autonomously in the cell. For example, the vector can be a plasmid with an origin of replication which is operable in eukaryote cells. In other embodiments, the gene expression cassette is stably integrated into the genome of the cell. In a preferred aspect, the recombinant cell further includes a second nucleic acid that comprises a gene expression cassette that includes a gene encoding a PXR or CAR, preferably human PXR or CAR, operably linked to a promoter. The gene expression cassette encoding the PXR or CAR can be provided on a vector that can replicate autonomously in the cell. For example, the vector can be a plasmid with an origin of replication which is operable in eukaryote cells, e.g., the SV40 origin of replication. In other embodiments, the gene expression cassette encoding the PXR or CAR is stably integrated into the genome of the cell.

The recombinant cells are grown in tissue culture dishes or wells of a tissue culture plate in medium containing an analyte being tested for ability to induce expression of CYP3A4 for a time sufficient for the cells to produce detectable reporter gene product based upon a positive control in which the recombinant cells are incubated in the presence of a known CYP3A4 inducer. In the case of transiently transfected cells, 48 hours is usually sufficient. Afterwards, expression of the reporter gene is determined either by measuring the amount of reporter gene product produced or the activity of the reporter gene on a substrate.

In the case of a reporter gene product that is secreted by the cell or bound to the outer membrane of the cell, the medium is removed and analyzed for the reporter gene product or a substrate for the reporter gene product is provided and activity of the reporter gene on the product is measured. SEAP is an example of a secreted reporter gene product in which its expression is determined by measuring its activity on a labeled substrate. Placental alkaline phosphatase (PLAP) is an example of reporter gene product which is bound to the outer cell membrane. In the case of a reporter gene which is not secreted and not bound to the outer membrane, expression can be determined by harvesting the cells from the tissue culture plates, lysing the cells, and either measuring the amount of reporter gene product made or measuring activity of the reporter gene product on a substrate. Alternatively, the recombinant cells are provided a substrate for the reporter gene product which is taken up by the cell and measuring activity of the reporter gene on the substrate taken up by the cell. An example is the β-lactamase-based reporter system and substrates disclosed in U.S. Pat. Nos. 6,472,205, 6,291,162, 5,955,604, and 5,741,657, and WO9630540, all to Tsien et al.

While it is currently preferred that the reporter gene be the SEAP gene, other embodiments of the present invention, the reporter gene can be the green fluorescence protein (GFP) gene, uroporphyrinogen III methyltransferase (cobA) gene, β-galactosidase (LacZ) gene, β-glucoronidase (Gluc) gene, β-lactamase (BLA) gene, chloramphenicol acetyl transferase (CAT) gene, luciferase, PLAP gene, or the like.

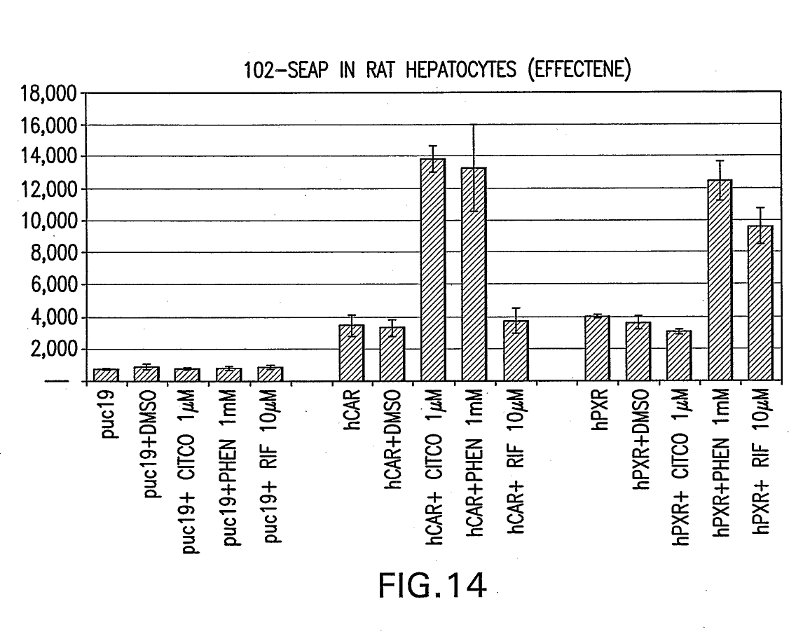

No simple cell-based assay for identifying analytes that activate CYP3A4 expression via CAR is believed to be currently available in the art because in most of the cell lines that have been analyzed, CAR appears to be constitutively activated and in many cells such as hepatocyte cell lines it is not possible to distinguish the contribution of CAR to activation of CYP3A4 from the contribution of PXR. We have discovered that primary cultures of rat hepatocytes cotransfected with clone 102-SEAP and a gene expression cassette encoding either PXR or CAR can distinguish between analytes that activate CYP3A4 expression via interactions with PXR from analytes that activate CYP3A4 expression via interactions with CAR. For example, as shown in FIG. 14 and described in Example 6, a primary culture of rat hepatocytes was transfected with the reporter gene expression cassette, clone 102-SEAP, or cotransfected with a gene expression cassette encoding the human CAR. When clone 102-SEAP was transfected into the primary rat hepatocytes alone and the transfected cells treated with compounds known to be inducers of CAR, there was no significant induction of the reporter gene. When clone 102-SEAP was cotransfected into the primary rat hepatocytes with a gene expression cassette encoding the human CAR, there was some constitutive CAR activation. However, compounds known to induce CYP3A4 activity via CAR were able to induce significant expression of the reporter gene via the cotransfected human CAR (FIG. 14). When clone 102-SEAP was cotransfected into the primary rat hepatocytes with a gene expression cassette encoding the human PXR, here again there was some constitutive PXR activation. However, compounds known to induce CYP3A4 activity via PXR were able to induce significant expression of the reporter gene (FIG. 14). The results indicated that expression of the reporter gene was driven solely by interaction of the compound with the cotransfected human PXR or CAR and the interaction was specific for the receptor.

As shown in FIG. 9, induction of expression of the reporter gene in clone 102-SEAP was about 23-fold over background. It is believed the 23-fold inducibility of the promoter was an important factor in detecting PXR and CAR expression in the primary hepatocytes. Composite promoters comprising other arrangements of PXR binding sites but with similar or greater inducibility compared to the composite promoter of clone 102-SEAP could also be used in the above assays using primary hepatocytes. Therefore, the present invention further provides methods for identifying analytes that activate CYP3A4 expression solely through interactions with CAR, analytes that activate CYP3A4 expression solely through interactions with PXR, and analytes that can activate CYP3A4 expression through interactions with either PXR or CAR.

To identify analytes that activate CYP3A4 expression via interactions with CAR, a primary culture of hepatocytes, preferably rat hepatocytes, is cotransfected with a reporter gene expression cassette as disclosed herein, preferably a reporter gene expression cassette wherein the reporter gene is operably linked to the composite promoter of clone 102-SEAP or a composite promoter comprising the nucleotide sequence of SEQ ID NO:18 operably linked to a heterologous promoter, and a gene expression cassette encoding CAR, preferably a human CAR, to produce a culture of recombinant cells. The cells are transfected either in batch and plated to tissue culture dishes or wells of a multiple-well tissue culture dish or transfected when already attached to the surface of tissue culture dishes or wells. In either case, after incubating the recombinant cells in a growth medium for a time sufficient to allow for uptake of the gene expression cassettes encoding the reporter gene and CAR, the recombinant cells are then incubated in a medium containing an analyte to be tested.

After incubating the recombinant cells for a time sufficient to allow expression of the reporter gene via activation of CAR as can be determined in a positive control in which recombinant cells are incubated in the presence of a known inducer of CYP3A4 activity via CAR, expression of the reporter gene is determined. It is desirable to include a negative control which does not contain an analyte but which contains the vehicle for the analyte. In the case of a reporter gene encoding SEAP, expression can be determined by measuring activity of the SEAP in an aliquot of the culture medium. In a preferred embodiment, the composite promoter operably linked to the reporter gene is the composite promoter comprising clone 102-SEAP.

To identify analytes which activate CYP3A4 via PXR, the above cotransfection is performed using a gene expression cassette encoding PXR and not the CAR. Preferably, the PXR is a human PXR. The positive control comprises incubating an aliquot of the recombinant cells with a known inducer of CYP3A4 via PXR.

To identify analytes which activate CYP3A4 solely via an interaction with PXR or CAR or which activate CYP3A4 via PXR or CAR, cultures of recombinant cells from each of the above cotransfections are provided. The same analyte is incubated with recombinant cells from each of the cotransfections followed by detection of expression of the reporter gene. An analyte that activates CYP3A4 expression via solely through an interaction PXR will express the reporter gene only in the culture of recombinant cells comprising the gene expression cassette encoding PXR. An analyte that activates CYP3A4 expression via solely through an interaction CAR will express the reporter gene only in the culture of recombinant cells comprising the gene expression cassette encoding CAR. An analyte that activates CYP3A4 expression via through an interaction with either PXR or CAR will express the reporter gene in both cultures.

Any one of the above can be adapted to screen a plurality of analytes at a time. For example, a quantity of cells sufficient for a multiplicity of cultures are transfected with the reporter gene expression cassette. Aliquots of the transfection are separately plated to tissue culture dishes or wells of a multiple-well tissue culture dish and incubated for a time sufficient to allow the recombinant cells to adhere to the surface of the dish or well and uptake of the reporter gene expression cassette. Each aliquot of plated recombinant cells is then incubated in a medium containing an analyte to be tested. Preferably, positive and negative controls are provided. After incubating the recombinant cells for a time sufficient to allow expression of the reporter gene via activation of PXR or CAR as can be determined in the control, expression of the reporter gene is determined.

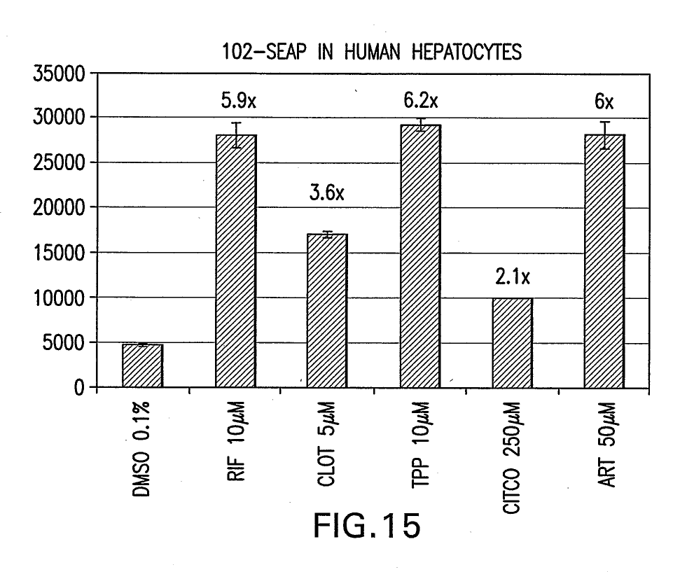

We further discovered that primary cultures of human hepatocytes transfected solely with the reporter gene expression cassette, clone 102-SEAP, and treated with compounds known to induce CYP3A4 activity via interactions with PXR or CAR, were responsive to the compounds (See Example 7 and FIG. 15). The results suggested human hepatocytes transfected with a reporter gene cassette comprising a composite promoter as disclosed herein, preferably, a composite promoter with a similar or greater fold of inducibility compared to the composite promoter of clone 102-SEAP, provide a simple screening assay for identifying analytes that activate CYP3A4 expression.

Therefore, the present invention further provides a method for identifying analytes that activate CYP3A4 expression via interactions with PXR or CAR comprising transfecting a primary culture of hepatocytes, preferably human hepatocytes, with a reporter gene expression cassette as disclosed herein, preferably, a reporter gene operably linked to the composite promoter of clone 102-SEAP or a composite promoter comprising the nucleotide sequence of SEQ ID NO:18 operably linked to a heterologous promoter, to produce a culture of recombinant cells. The cells are transfected either in batch and plated to tissue culture dishes or wells of a multiple-well tissue culture dish or transfected when already attached to the surface of tissue culture dishes or wells. In either case, after incubating the recombinant cells in a growth medium for a time sufficient to allow for uptake of the reporter gene expression cassette, the recombinant cells are then incubated in a medium containing an analyte to be tested. After incubating the recombinant cells for a time sufficient to allow expression of the reporter gene via activation of PXR or CAR as can be determined in a positive control in which recombinant cells are incubated in the presence of a known inducer of CYP3A4 activity via PXR or CAR, expression of the reporter gene is determined. It is desirable to include a negative control which does not contain an analyte but which contains the vehicle for the analyte. In the case of a reporter gene encoding SEAP, expression can be determined by measuring activity of the SEAP in an aliquot of the culture medium. In a preferred embodiment, the composite promoter operably linked to the reporter gene is the composite promoter comprising clone 102-SEAP.

The method can be adapted to screen a plurality of analytes at a time. For example, a quantity of cells sufficient for a multiplicity of cultures are transfected with the reporter gene expression cassette. Aliquots of the transfection are separately plated to tissue culture dishes or wells of a multiple-well tissue culture dish and incubated for a time sufficient to allow the cells to adhere to the surface of the dish or well and uptake of the reporter gene expression cassette. Each aliquot of plated recombinant cells is then incubated in a medium containing an analyte to be tested. Preferably, at least one aliquot is incubated with a known inducer of CYP3A4 activity via PXR or CAR to serve as a positive control. It is also desirable to include a negative control consisting of the vehicle for the analytes as well. After incubating the recombinant cells for a time sufficient to allow expression of the reporter gene via activation of PXR or CAR as can be determined in the control, expression of the reporter gene is determined.

The method of the present invention is particularly useful for high throughput screening (HTS) of analytes to identify analytes which can mediate induce CYP3A4 expression. Often chemical entities with useful properties are generated by identifying a chemical compound (called a “lead compound”) with some desirable property or activity, creating variants of the lead compound, and evaluating the property and activity of those variant compounds. The current trend is to shorten the time scale for all aspects of drug discovery. Because of the ability to test large numbers quickly and efficiently, high throughput screening (HTS) methods are replacing conventional lead compound identification methods.

In one aspect, high throughput screening methods involve providing a library containing a large number of drug candidates. Such “combinatorial chemical libraries” are then screened in one or more assays, to identify those library members particular chemical species or subclasses that display a desired characteristic activity. The compounds thus identified can serve as conventional “lead compounds”.

Devices for the preparation of combinatorial libraries are commercially available (See, for example, 357 MPS, 390 MPS, Advanced Chem Tech, Louisville, Ky.; Symphony, Rainin, Woburn, Mass.; 433A Applied Biosystems, Foster City, Calif.; 9050 Plus, Millipore, Bedford, Mass.). A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (See, for example, ComGenex, Princeton, N.J.; Asinex, Moscow, Russia; Tripos, Inc., St. Louis, Mo.; ChemStar, Ltd, Moscow, Russia; 3D Pharmaceuticals, Exton, Pennsylvania; Martek Biosciences, Columbia, Md.).

Any of the assays described herein are amenable to high throughput screening. As described above, the analytes are preferably screened by the methods disclosed herein. High throughput systems for such screening are well known to those of skill in the art. Thus, for example, U.S. Pat. No. 5,559,410 discloses high throughput screening methods for protein binding, while U.S. Pat. Nos. 5,576,220 and 5,541,061 disclose high throughput methods of screening for ligand/antibody binding.

In addition, high throughput screening systems are commercially available (See, for example, Zymark Corp., Hopkinton, Mass.; Air Technical Industries, Mentor, Ohio; Beckman Instruments, Inc. Fullerton, Calif.; Precision Systems, Inc., Natick, Mass.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols. Thus, for example, Zymark Corp. provides technical bulletins describing screening systems for detecting the modulation of gene transcription, ligand binding, and the like.

The present invention further provides a kit for determining whether an analyte is an inducer of CYP3A4, which comprises a container that contains a nucleic acid comprising a reporter gene operably linked to a composite promoter. Preferably, the reporter gene encodes SEAP. It is further preferable that the composite promoter comprise a composite enhancer element comprising at least two different enhancer elements or PXR/CAR binding sites (for example, at least one each of dDR3 and dER6), operably linked to a minimal promoter. In further still embodiments, it is preferable that the minimal promoter is the CMV minimal promoter. In further still embodiments of the kit, the kit further includes a second container which contains a second nucleic acid comprising a gene encoding PXR or CAR or both, preferably a human PXR or CAR. The kit can further still include one or more additional containers which contain reagents for transfecting cells. Further still or in the alternative, the kit can include one or more containers which contain reagents for detecting the reporter gene or measuring activity of the reporter gene or both.

The following examples are intended to promote a further understanding of the present invention.

### EXAMPLE 1

To determine whether a PXR-responsive promoter that was stronger than that in the prior art could be constructed, the nucleotide sequence of the responsive cis-acting nucleotide elements of the CYP3A4 promoter were chemically synthesized into short oligomers, which were then randomly assembled into composite promoters to produce recombinant libraries consisting of a plurality of composite promoter configurations, which were then screened for transcriptional activity. The strategy is shown in FIG. 2B. A similar strategy had been used by Li et al. Nature Biotech. 17: 241-245 (1999) to generate a composite muscle specific promoter that was stronger than the naturally occurring myogenic promoters.

The nucleic acid manipulations were performed in accordance with standard molecular biology methods such as those described in Sambrook et al., Molecular Cloning: A Laboratory Manual 2nd Edition; Cold Spring Harbor Laboratory Press, Cold Spring Harbor, N.Y., (1989) or Sambrook and Russell, Molecular Cloning: A Laboratory Manual, 3rd Edition. Cold Spring Harbor Laboratory Press, Plainview, N.Y. (2001)). Plasmid DNA was prepared from overnight cultures in LB broth using plasmid purification columns (Qiagen, Valencia, Calif.) according to manufacturer's instructions.

The pER6, dDR3, and dER6 enhancer elements were selected because of their ability to bind human PXR. The strategy for the construction of a PXR-responsive synthetic promoter is depicted in FIG. 2B. Specific oligomers consisting of both strands of each of the three elements (pER6, dDR3 and dER6) plus a few nucleotides at the edges were generated.

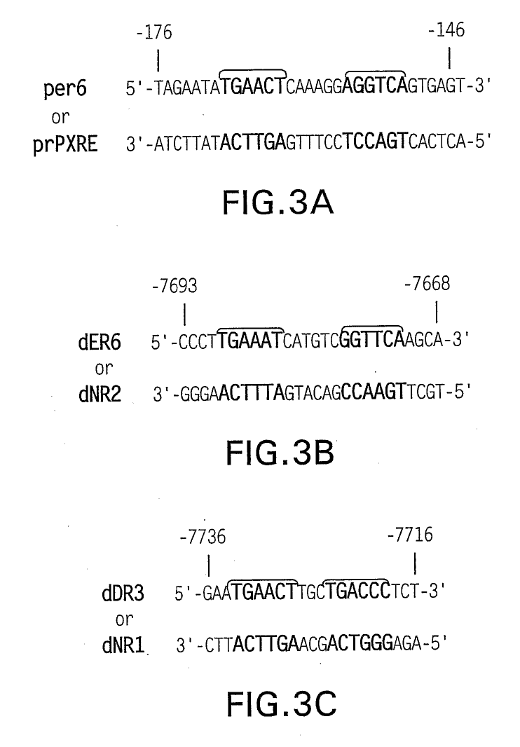

A complementary pair of single-stranded oligomers corresponding to pER6 from nucleotides −176 to −146 of the CYP3A4 promoter were synthesized. The forward oligomer was 5′-TAG AAT ATG AAC TCA AAG GAG GTC AGT GAG T-3′ (SEQ ID NO:2) and the complementary oligomer was 5′-ACT CAC TGA CCT CCT TTG AGT TCA TAT TCT A-3′ (SEQ ID NO:3). The double-stranded pER6 is shown in FIG. 3A.

A complementary pair of single-stranded oligomers corresponding to dDR3 from −7736 to −7716 of the CYP3A4 promoter were synthesized. The forward oligomer was 5′-GAA TGA ACT TGC TGA CCC TCT-3′ (SEQ ID NO:5) and the complementary oligomer was 5′-AGA GGG TCA GCA AGT TCA TTC-3′ (SEQ ID NO:6). The double-stranded dDR3 is shown in FIG. 3B.

A complementary pair of single-stranded oligomers corresponding to dER6 from −7693 to −7668 of the CYP3A4 promoter were synthesized. The forward oligomer was 5′-CCC TTG AAA TCA TGT CGG TTC AAG CA-3′ (SEQ ID NO:8) and the complementary oligomer was 5′-TGC TTG AAC CGA CAT GAT TTC AAG GG-3′ (SEQ ID NO:9). The double-stranded dER6 is shown in FIG. 3C.

The complementary single-stranded oligomers for each of the oligomer pairs were phosphorylated at the 5′ end with T4 polynucleotide kinase. Then, the complementary single-stranded oligomers for each of the oligomer pairs were allowed to anneal to form a double-stranded duplexes of the enhancer element. Then, equal amounts of each of the three double-stranded duplexes were added to a ligation mixture containing 6000 units of DNA ligase in a random ligation reaction. After allowing the ligation reaction to proceed for 16 hours at 16° C., the ligation reaction was applied to an agarose gel and electrophoresed at Vs. DNA size markers were included on the gel. The region of the agarose gel corresponding to the region shown by the markers to contain a population of double-stranded DNA fragments from between about 150 to about 350 bp (about 5 to 15 copies of enhancer elements per DNA fragment) was cut from the gel and the DNA eluted from the gel using JETSORB by Genomed, GmbH, Lohne, Germany, according to the manufacturer's instructions.

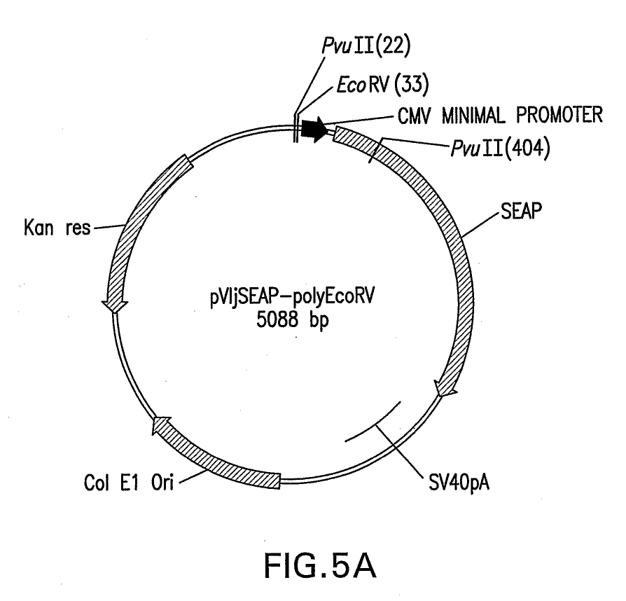

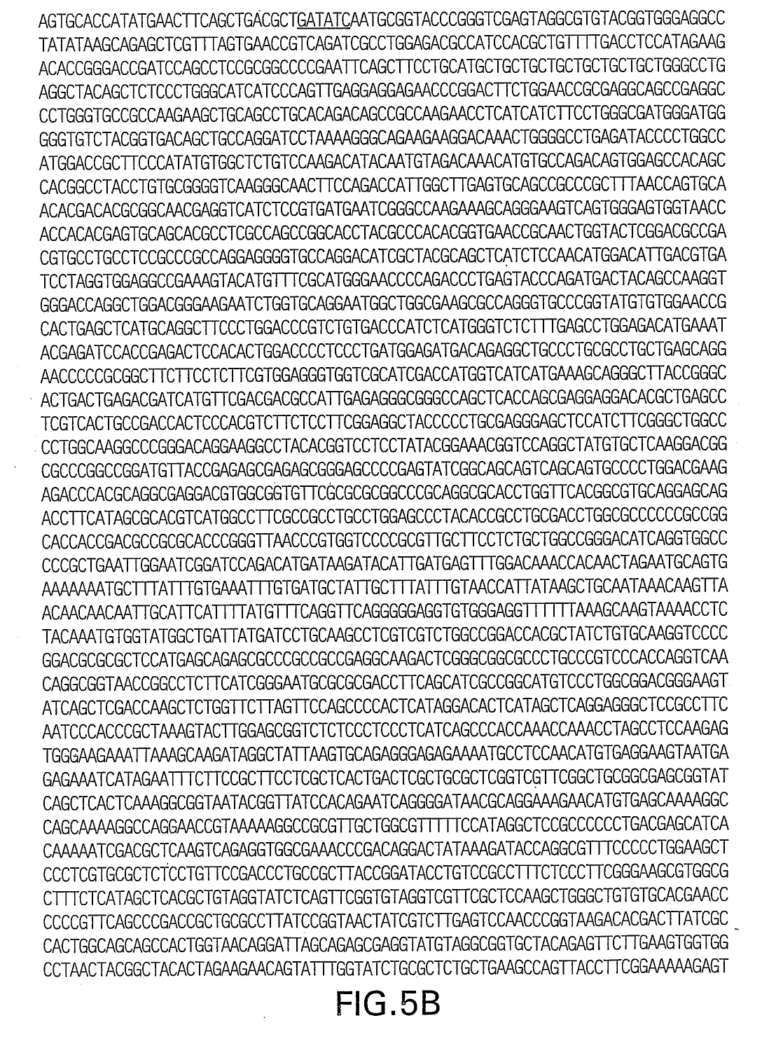

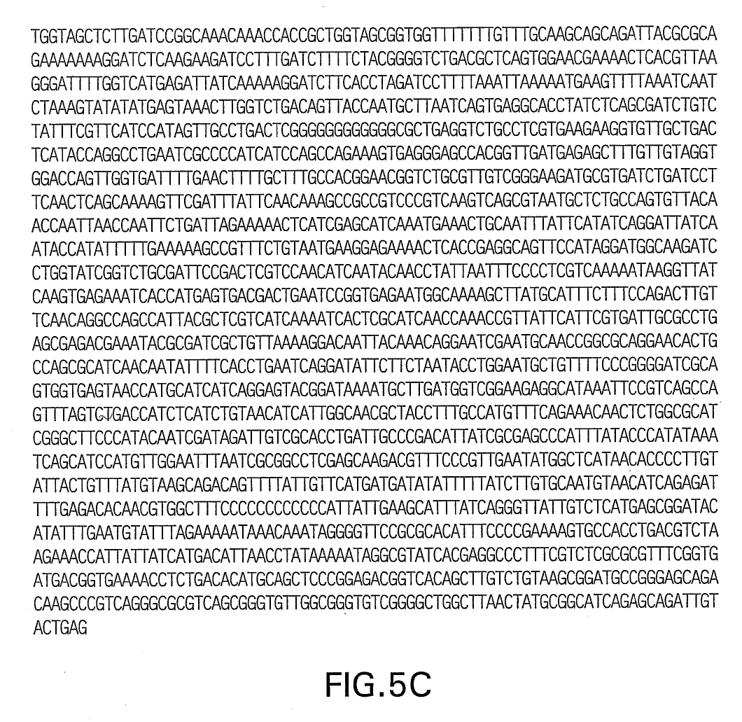

The eluted double-stranded DNA fragments were blunt-ligated into the EcoRV site of the plasmid, pVIj-SEAP-polyEcoRV, upstream of the 120 bp CMV minimal promoter operably linked to a 1521 bp nucleic acid encoding the SEAP reporter gene followed by a 346 bp SV40 poly adenylation (SV40pA) sequence to produce a plurality of plasmids, pVIj-cpr-SEAP (wherein “cpr” is “composite promoter”), each comprising one or more of the eluted double-stranded DNA fragments in any order and any combination to make a composite promoter which is operably linked to the CMV minimal promoter. The plasmid, pVIj-SEAP, (FIG. 5A) has been described in Salucci et al., Gene Therapy 9: 1415-1421 (2002) and Rizzuto et al., Human Gene Therapy 11: 1891-1900 (2000). The nucleotide sequence of the pVIj-SEAP-EcoRV plasmid (SEQ ID NO:21) is shown in FIG. 5B. Plasmid pBVIj-SEAP was derived from the pV1J plasmid described in Montgomery et al., DNA Cell Biol. 12: 777-783 (1993)). E. coli were transformed with the above plasmids containing the reporter gene cassettes comprising the composite promoters to produce a recombinant library comprising a plurality of clones, each clone comprising a composite promoter in a particular configuration. An aliquot of the library was plated onto agar plates to produce colonies. Several hundred colonies were screened by PCR using PCR primers flanking the EcoRV site to identify clones containing plasmids with about five copies of any enhancer element (data not shown). More than a hundred clones were selected for the functional test in HepG2 cells.

### EXAMPLE 2

Each of the cloned DNAs isolated from the colonies of the library in Example 1 was separately co-transfected with plasmid DNA encoding the human PXR into HepG2 cells as shown below to produce transiently transfected HepG2 cells, each expressing SEAP operably linked to one of the composite promoters. Expression of SEAP was measured in the presence and absence of 10 μM Rifampicin as an inducer of PXR activation of SEAP transcription.

The human hepatoma cell line, HepG2, was grown in high glucose Dulbecco's modified Eagle medium (DMEM; Life Technologies, Bethesda, Md.) supplemented with 2 mM L-glutamine, 100 U/mL of Penicillin, 100°g/mL streptomycin, and 10% fetal bovine serum. Cells were sub-cultivated twice a week with a 1:5 split ratio. For the assay, the HepG2 cells were split and seeded into the wells of six-well tissue culture plates. The next day after seeding, the plated cells were transfected with a mixture of DNA consisting of 0.9 μg of library reporter plasmid DNA from the library or reference reporter plasmid, pZDCVS ΔCYP3A4/SEAP (ΔCYP3A4/SEAP), which contains ΔCYP3A4 promoter of the prior art operably linked to the SEAP reporter gene, and 0.1 μg of the nuclear receptor (NR) donor plasmid DNA encoding the human PXR gene (pSG5 dATG-hPXR) in Lipofectamine and PLUS Reagent according to the protocol suggested by the manufacturer (Life Technologies, Bethesda, Md.). Three hours post-transfection, the medium for each well was replaced with fresh DMEM-GM containing 10 μM Rifampicin and the assays for SEAP expression were performed with Tropix Phospha-Light system kit (Applied Biosystems, Foster City, Calif.). Control assays consisted of 0.9 μg of the library reporter plasmid DNA and not NR donor plasmid DNA.

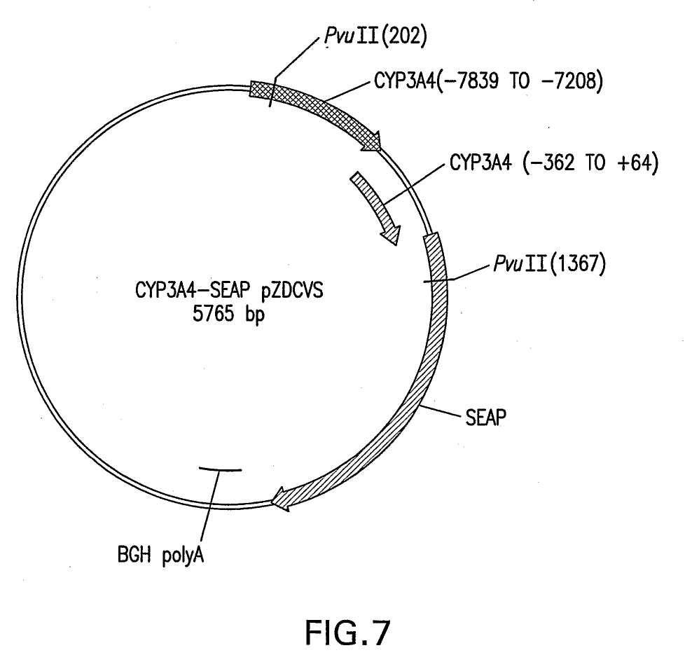

A diagram of the reference reporter plasmid, (ΔCYP3A4/SEAP), is shown in FIG. 7. The plasmid comprises the pZDCS plasmid (plasmid pZDCS was made by Dr. Yves Durocher at the Biotechnology institute of Canada, the construction of which was described in part in Durocher et al., Anal. Biochem. 284: 316-326 (2000)) in which the 1521 bp nucleic acid encoding SEAP operably linked to a composite promoter consisting of a first DNA fragment from −7839 to −7208 of the CYP3A4 promoter containing dDR3 and dER6 and a second DNA fragment from −362 to +64 of the CYP3A4 promoter containing pER6. The SEAP gene was followed by the 215 bp bovine growth hormone (BGH) polyA sequence. Plasmid ΔCYP3A4/SEAP was constructed as follows. The SEAP gene was removed from pSEAP-BASIC (Clontech, Palo Alto, Calif.) and inserted into pcDNA3 (Invitrogen). Then the SEAp gene was removed and cloned into pZeoSV2 (Invitrogen, San Diego, Calif.) to produce plasmid pZeo/SEAP. The SV40 enhancer-promoter region was removed from pZeo/SEAP to produce plasmid pZDCVS. Plasmid pZDCVS was then digested with HindIII and NheI and the following CYP3A4 genomic fragments, which had been PCR amplified from human genomic DNA with PCR primers designed to produce the first DNA (nucleotides −7839 to −7208) of the CYP3A4 promoter flanked by NheI and BglII sites and the second DNA fragment (nucleotides −362 to +64 of the CYP3A4 promoter) flanked by BglII and HindIII sites, were inserted between the HindIII and NheI sites of the digested pZDCVS to produce plasmid ΔCYP3A4/SEAP. A similar ΔCYP3A4 promoter in ΔCYP3A4/SEAP has also been described by Goodwin et al. in Molec. Pharma. 56: 1329-1339 (1999).

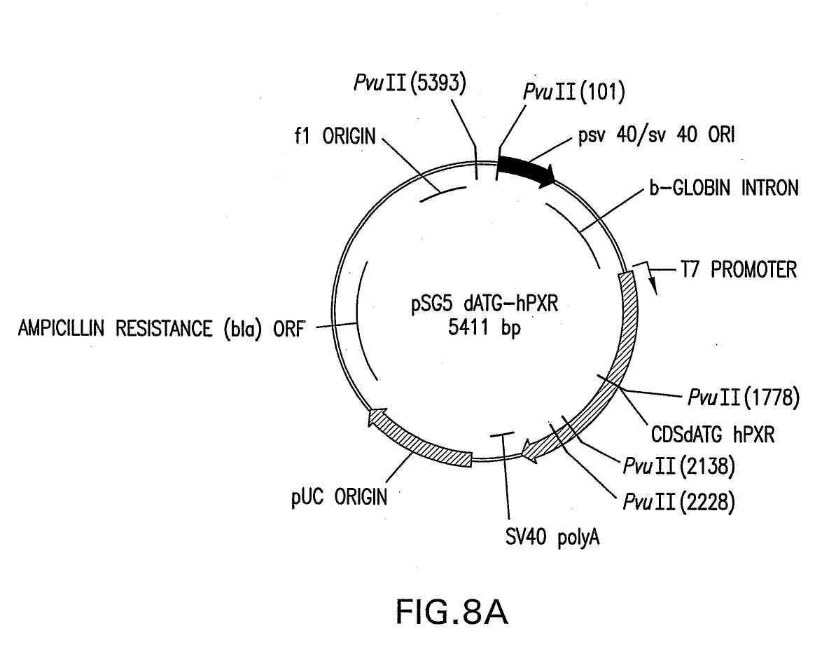

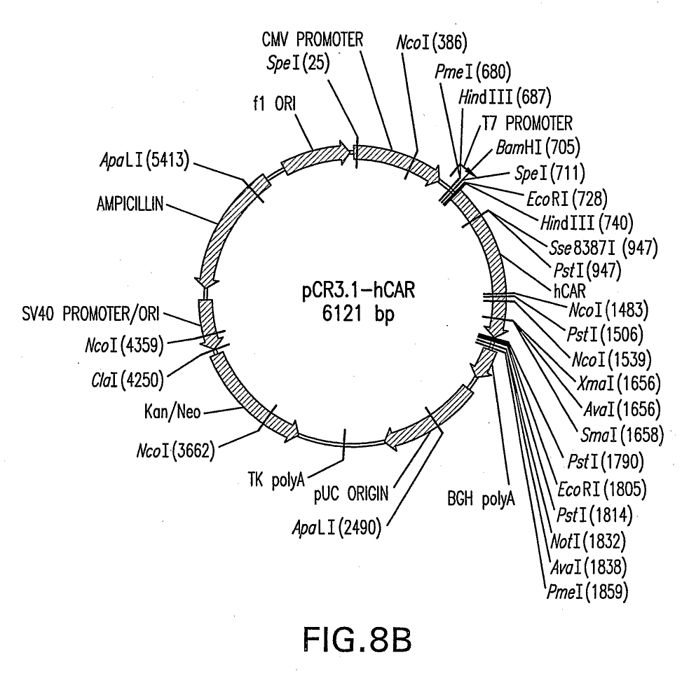

A diagram of the NR donor plasmid encoding the human PXR, pSG5 dATG-hPXR, is shown in FIG. 8A. The plasmid comprises the pSG5 plasmid, which is obtainable from Stratagene, La Jolla, Calif., and a 1335 bp nucleic acid encoding the human PXR (hPXR) with the ATG start codon deleted inserted into the multiple cloning site of the plasmid located between the 573 bp human beta-globin first intron operably linked to the 439 bp SV40 promoter SV40 origin and the 134 bp SV40 polyadenylation site. The DNA encoding the human PXR was PCR amplified from a human fetal liver cDNA library. The 5′ PCR primer that was used included an ATG initiation codon in place of the Leu initiation codon shown in the published sequence for human PXR. The PCR DNA product encoding the human PXR was cloned into the TA cloning vector (Invitrogen). The DNA encoding the human PXR, which was flanked by EcoRI sites provided by the TA cloning vector, was removed from the TA cloning vector by digesting with EcoRI. The DNA fragment was then inserted into the EcoRI site of plasmid pSG5 to make plasmid pSG5 dATG-hPXR.

Several transfection experiments were performed to test more than a hundred of the selected clones. Despite a degree of variability in the fold induction of transcription of SEAP from the ΔCYP3A4 promoter (ranging from 2 to 7 fold), likely due to status of the cells, the relative signal ratios between the induction of transcription of SEAP from the composite promoters of the various library clones versus induction of transcription from the ΔCYP3A4 promoter was always maintained.

Results from a typical screening assay is shown in FIG. 9. As shown, transcription of SEAP from the reference plasmid, ΔCYP3A4/SEAP, was induced about six-fold in the presence of Rifampicin while transcription of SEAP from several of the library plasmids were induced to higher levels. For example, induction of transcription of SEAP from the composite promoter of clone 102-SEAP from the library was consistently from between about four to five times better than induction of transcription of SEAP from the ΔCYP3A4 promoter (23-fold induction versus six-fold induction, respectively) and gave a much greater overall SEAP signal. It was interesting to note that expression of SEAP in cells transfected with some of the clones from the library was induced to a higher level than the expression of SEAP from cells transfected with ΔCYP3A4/SEAP. For example, induction of SEAP transcription for clone 61 was about 11-fold; however, the overall SEAP signal was lower or similar to the signal from ΔCYP3A4/SEAP. These clones had a lower background expression in the presence of human PXR but a greater response with Rifampicin. Among the clones tested, clone 102-SEAP has been identified to give a high responsiveness to the CYP3A4 inducer Rifampicin, both in terms of fold induction and in terms of a strong and robust reporter (SEAP) signal. Based on the above, clone 102-SEAP was selected for further analysis against a panel of compounds.

The enhancer or binding site arrangements for several of the composite promoters of these clone are shown in FIG. 6. The arrangement of enhancers for several of the clones are shown in FIG. 6. The nucleotide sequences for the clones shown in FIG. 6 are as follows.

The nucleotide sequence from Clone 26-SEAP comprising the PXR binding sites inserted upstream of the minimal CMV promoter is 5′-TAGAATATGAACTCAAAGGAGGTCAGTGAGT TAGAATATGAACTCAAAGGAGGTCAGTGAGTCCCTTGAAATCATGTCGGTTCAAGCA-3′ (SEQ ID NO:14). In the order shown in FIG. 6, the sequence comprises from 5′ to 3′, two prER6 elements followed a dER6 element.

The nucleotide sequence from Clone 33-SEAP comprising the PXR binding sites inserted upstream of the minimal CMV promoter is 5′-TGCTTGAACCGACATGATTTCAAGGG AGAGGGTCAGCAAGTTCATTCTAGAATATGAACTCAAAGGAGGTCAGTGAGT GAATGAACTTGCTGACCCTCT GAATGAACTTGCTGACCCTCT ACTCACTGACCTCCTTTGAGTTCATATTCTA-3′ (SEQ ID NO:15). In the order shown in FIG. 6, the sequence comprises from 5′ to 3′, a dER6 element, a dDR3 element, a prER6, two dDR3 elements, and a prER6 element.

The nucleotide sequence from Clone 61-SEAP comprising the PXR binding sites inserted upstream of the minimal CMV promoter is 5′-AGAGGGTCAGCAAGTTCATTC AGAGGGTCAGCAAGTTCATTC TGCTTGAACCGACATGATTTCAAGGG AGAGGGTCAGCAAGTTCATTC TAGAATATGAACTCAAAGGAGGTCAGTGAGT AGAGGGTCAGCAAGTTCATTC-3′ (SEQ ID NO:16). In the order shown in FIG. 6, the sequence comprises from 5′ to 3′, two dDR3 elements, a dER6 element, a dDR3 element, a prE6 element, and a dDR3 element.

The nucleotide sequence from Clone 71-SEAP comprising the PXR binding sites inserted upstream of the minimal CMV promoter is 5′-TGCTTGAACCGACATGATTTCAAGGG CCCTTGAAATCATGTCGGTTCAAGCA ACTCACTGACCTCCTTTGAGTTCATATTCTA CCCTTGAAATCATGTCGGTTCAAGCA AGAGGGTCAGCAAGTTCATTC TGCTTGAACCGACATGATTTCAAGGG TGCTTGAACCGACATGATTTCAAGGG AGAGGGTCAGCAAGTTCATTC ACTCACTGACCTCCTTTGAGTTCATATTCTA GAATGAACTTGCTGACCCTCT ACTCACTGACCTCCTTTGAGTTCATATTCTA-3′ (SEQ ID NO:17). In the order shown in FIG. 6, the sequence comprises from 5′ to 3′, two dER6 elements, a prER6 element, a dER6 element, a dDR3 element, two dER6 elements, a dDR3 element, a prER6 element, and a prER6 element.

The nucleotide sequence from Clone 102-SEAP comprising the PXR binding sites inserted upstream of the minimal CMV promoter is 5′-AGAGGGTCAGCAAGTTCATTC TGCTTGAACCGACATGATTTCAAGGG AGAGGGTCAGCAAGTTCATTC GAATGAACTTGCTGACCCTCT GAATGAACTTGCTGACCCTCT-3′ (SEQ ID NO:18). In the order shown in FIG. 6, the sequence comprises from 5′ to 3′, a dDR3 element, a dER6 element, and two dDR3 elements. The sequence does not contain a prER6 element.

### EXAMPLE 3

HepG2 cells transfected with clone 102-SEAP DNA were evaluated for ability to identify CYP3A4 inducers.

The human hepatoma cell line, HepG2, grown as in Example 2 were split and seeded into the wells of six-well tissue culture plates. The next day after seeding, the plated cells were transfected with a mixture of DNA consisting of 0.9 μg of clone 102-SEAP DNA or ΔCYP3A4/SEAP DNA and 0.1 μg pSG5 dATG-hPXR DNA in Lipofectamine and PLUS Reagent. Control assays consisted of 0.9 μg of clone 102-SEAP DNA and not the NR donor plasmid.

Three hours post-transfection, the medium for each well was replaced with fresh DMEM-GM containing either 200 μM Omeprazole, 10 μM Androstanol, 100 μM Cholic Acid, 6 μM Clotrimazole, 125 nM Hyperforin, 12.5 μM Lovastatin, 100 μM N-propyl-p-hydroxy-benzoate, 1 mM Phenobarbital, 10 μM Rifampicin, or 10 μM RU486. After incubating the cells 48 hours at 37° C., the medium form wells were collected for SEAP assays. The assays for SEAP expression were performed with Tropix Phospha-Light system kit.

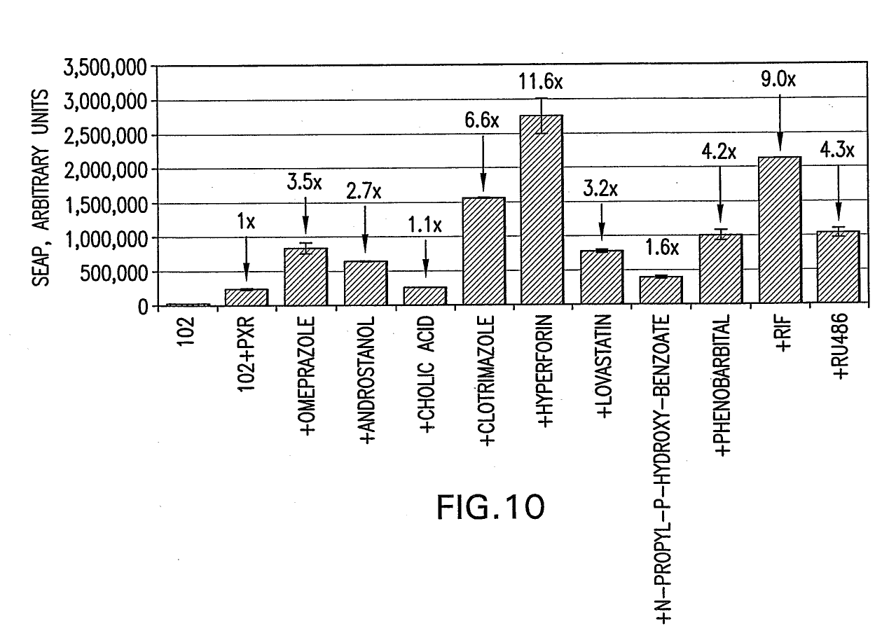

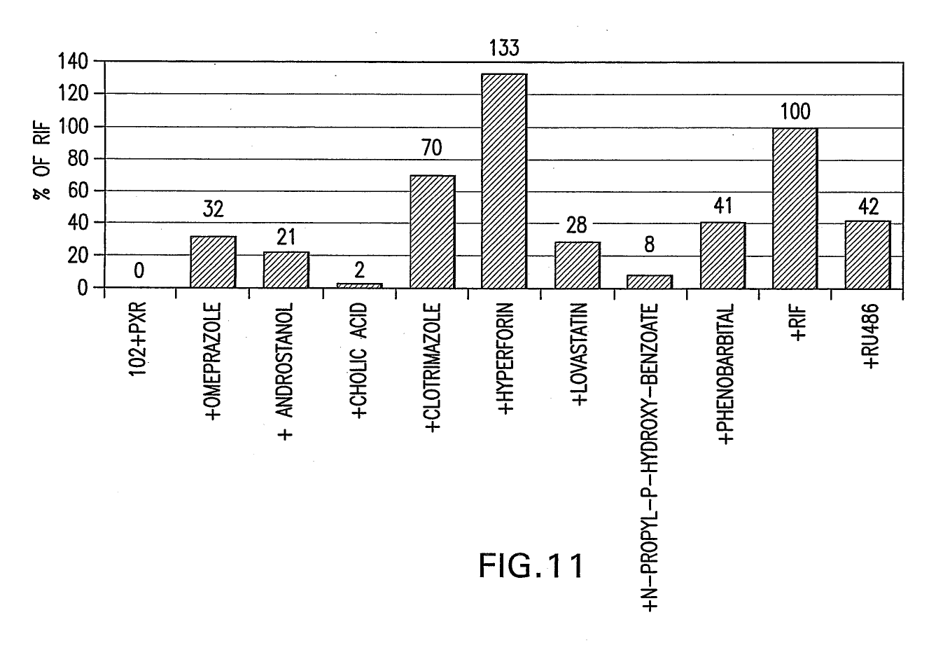

The results are shown in FIGS. 10 and 11. FIG. 10 shows the fold induction of SEAP transcription by the various compounds. FIG. 11 shows the fold induction of SEAP transcription expressed as a percentage of the induction by rifampicin. The response of clone 102-SEAP to transcription induction by these compounds was in agreement to the published results both in human hepatocytes and in HepG2/CYP3A4 induction assays (Luo et al., Drug Metab. Disp. 30: 795-804 (2002); Moore et al., J. Biol. Chem. 275: 15122-15127 (2002); Raucy, Drug. Metab. Disp. 31: 533-539 (2003)). The comparison with the reference ΔCYP3A4/SEAP plasmid (data not shown) revealed that the reference reporter plasmid behaves in similar manner but that clone 102 consistently gives a much higher signal.

Though the prER6, dER6, dDR3 enhancer sites are located at different distances from each other in the native CYP3A4 promoter, about 8 kb upstream of the transcription start site for dER6 and dDR3, the results shown herein indicate that the ability of dER6 and dDR3 to enhance transcription is distance-independent and that their functional effects are additive. Even though it is very difficult to rationalize the effect of distribution and orientation of the different binding sites in the clones and in clone 102-SEAP in particular, it can be speculated that the multimerization of dDR3, and perhaps the right orientation, might play a major role in conferring a potent response to rifampicin and other PXR ligands. This is in agreement with the higher affinity of the human PXR/human RXR complex for this site with respect to dER6 and pER6 (Goodwin et al., Mol. Pharmcol. 56: 1329-1339 (1999)). One concern relative to the use of an artificial promoter that contains only binding sites for PXR is that the native CYP3A4 promoter may reproduce responses that more faithfully mirror those obtained in vivo. However, this objection is easily overcome by the fact that performing the HepG2 assay without hPXR, neither the reference reporter plasmid CYP3A4/SEAP nor clone 102-SEAP responded to Rifampicin. Both reporter plasmids were strictly dependent on the co-transfection of hPXR.

The composite promoters, particularly that exemplified by clone 102-SEAP, clearly offer the advantage of very high signal and high responsiveness to CYP3A4 inducers. In comparison with the reference reporter plasmid CYP3A4/SEAP, the composite promoters, particularly as exemplified by clone 102-SEAP, are amenable for the use in in-vitro high-throughput screening of drug candidates and provide a much more robust assay.

### EXAMPLE 4

Because CAR is another nuclear receptor that recognizes similar sites in the promoters of many PXR responsive genes, clone 102-SEAP DNA was tested in co-transfections with a plasmid that expressed human CAR.

The NR donor plasmid encoding the human CAR was made as follows. The human CAR was made by RT-PCR from a human liver mRNA preparation. The PCR primers used were 5′-GAAGC TTGTT CATGG CCAGT AGGGA AGATG AGC-3′ (SEQ ID NO:19) AND 5′-TGGCC TCAGC TGCAG ATCTC CTGGA GC-3′ (SEQ ID NO:20). The RT-PCR conditions were 15 seconds at 94° C., 30 cycles of 94° C. for 30 seconds followed by 68° C. for 3 minutes, then 68° C. for 3 minutes, and storage at 4° C. A 1047 bp nucleic acid encoding the human CAR (hCAR) obtained from the RT-PCR was inserted into the TA cloning site of pCR3.1 (Invitrogen, La Jolla, Calif.) to make plasmid pCR3.1-hCAR (FIG. 8B). Expression of the hCAR was driven by the 596 bp CMV immediate early promoter.

The human hepatoma cell line, HepG2, grown as in Example 2 were split and seeded into the wells of six-well tissue culture plates. The next day, after seeding, the plated cells were transfected with a mixture of DNA consisting of 0.9 μg of clone 102-SEAP DNA or ΔCYP3A4/SEAP DNA and 0.1 μg DNA pCR3.1-hCAR in Lipofectamine and PLUS Reagent. Controls omitted the DNA encoding the human CAR. Additional controls consisted of DNA encoding the human PXR of the previous example.

Three hours post-transfection, the medium for each well was replaced with fresh DMEM-GM containing either 200 μM Omeprazole, 10 μM Androstanol, 100 μM Cholic Acid, 6 μM Clotrimazole, 125 nM Hyperforin, 12.5 μM Lovastatin, 100 μM N-propyl-p-hydroxy-benzoate, 1 mM Phenobarbital, 10 μM Rifampicin, or 10 μM RU486. After incubating the cells 48 hours at 37° C., the medium form wells were collected for SEAP assays. The assays for SEAP expression were performed with Tropix Phospha-Light system kit.

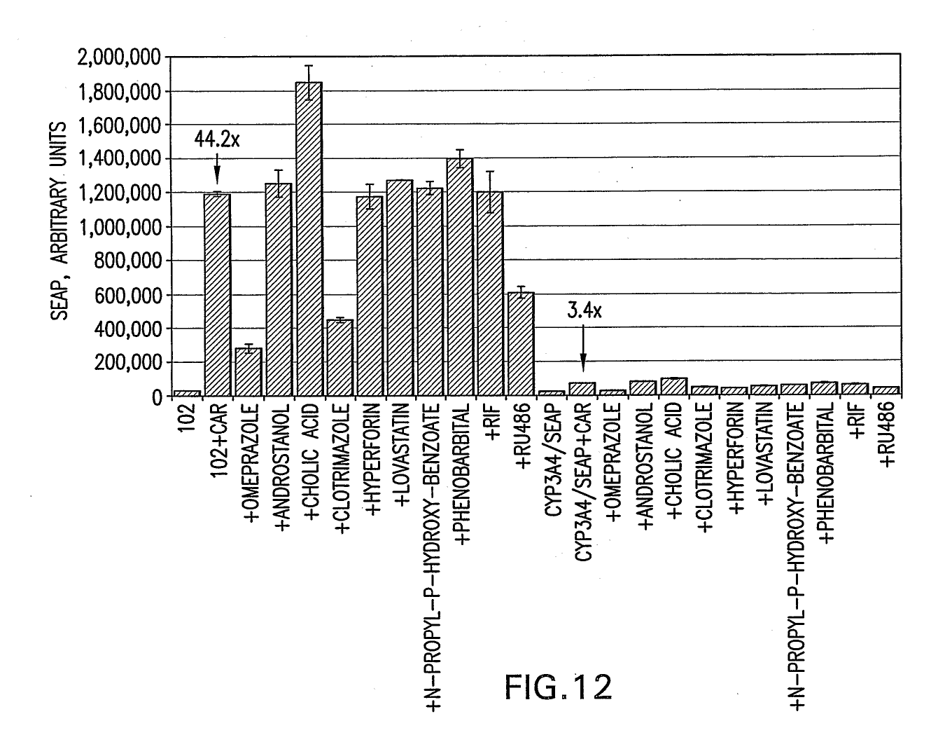

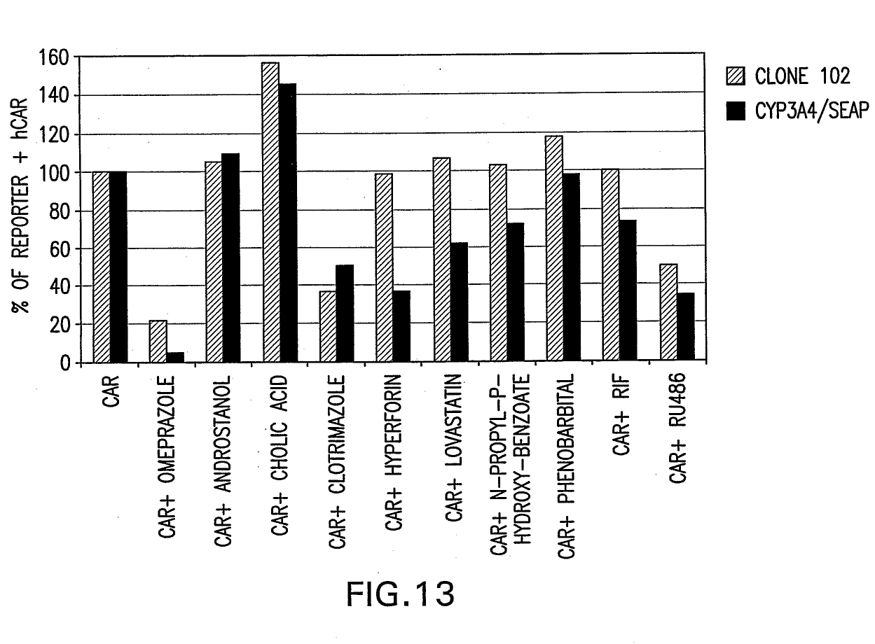

In contrast to the results in Example 3, co-transfection of a plasmid encoding human CAR into the HepG2 cells led to transcription activation of the ΔCYP3A4 promoter in the absence of any added compound. As shown in FIG. 12 (results expressed as SEAP arbitrary units) and in FIG. 13 (results expressed as percent of the maximal activation obtained by constitutive activation of transcription induced by human CAR), co-transfection of human CAR DNA with clone 102-SEAP DNA or ΔCYP3A4/SEAP DNA resulted in a 44.2-fold and 3.4 fold induction of SEAP transcription, respectively. The much higher induction of transcription from the composite promoter of clone 102-SEAP confirms that the composite promoter is more sensitive to nuclear receptor-mediated activation than the ΔCYP3A4 promoter.

Compounds, such as Clotrimazole and Omeprazole, already known as CAR repressors in vitro, clearly showed an inhibitory effect on transcription from both the clone 102-SEAP composite promoter and the ΔCYP3A4 promoter. Also, the antiprogestin, RU486, revealed to be an inhibitor of the constitutive activation of human CAR. In Moore et al., J. Biol. Chem. 275: 15122-15127 (2000) transcription from a reporter plasmid was induced 3.3-fold by human CAR whereas RU486 showed no inhibition effect in CV1 cells. For some other compounds, for example, Hyperforin, Lovastatin, and N-propyl-p-hydroxy-benzoate, the transcription from the composite promoter of clone 102-SEAP reporter plasmid was not affected while moderate inhibition of transcription from the ΔCYP3A4 promoter was observed (FIG. 13).

The high activation of transcription from the composite promoter of clone 102-SEAP by co-transfected human CAR might be explained by the greater affinity of the human CAR/human RXR complex for dDR3 (Goodwin et al., Mol. Pharmacol. 62: 359-365 (2002)). The composite promoters, particularly that exemplified by clone 102-SEAP, clearly offer the advantage of very high signal and high responsiveness to CYP3A4 inducers. In comparison with the reference reporter plasmid CYP3A4/SEAP, the composite promoters, particularly as exemplified by clone 102-SEAP, are amenable for the use in in-vitro high-throughput screening of drug candidates and provide a much more robust assay.

### EXAMPLE 5

A cell line containing a reporter gene expression cassette of the present invention can be made as follows.

The human hepatoma cell line, HepG2, is maintained in high glucose Dulbecco's modified Eagle medium (DMEM; Life Technologies, Bethesda, Md.) supplemented with 2 mM L-glutamine, 100 U/mL of Penicillin, 100 μg/mL streptomycin, and 10% fetal bovine serum. About 2×10⁵ cells are seeded into 100 mm tissue culture dishes and transfected with 1 μg of plasmid containing a gene expression cassette from Example 1 and the gene conferring neomycin resistance in Lipofectamine and PLUS Reagent according to the protocol suggested by the manufacturer (Life Technologies, Bethesda, Md.). Three hours post-transfection, the medium was replaced with fresh DMEM-GM. Twenty-four hours later, the cells are split and cultured in G418 selection DMEM (1 mg/mL G418). Culture medium is replaced every 3 days until colonies of cells are formed. Individual colonies are isolated and seeded into six-well plates. After the cells have grown to confluence, their ability to express SEAP in the presence of a known CYP3A4 activator is determined using the Tropix Phospha-Light system kit.

### EXAMPLE 6

This example demonstrates that cotransfecting the composite promoter and an expression vector encoding the hCAR or HPXR into fresh rat hepatocytes provides an assay that distinguishes CYP3A4 expression induced via analyte activated hCAR from expression induced via analyte activated hPXR.

Transfections and cotransfections of primary rat hepatocytes cells with EFFECTENE transfection reagent (Qiagen, Cat. No. 301425) in 24-well plates were performed as follows.

The day before transfection, hepatocytes were isolated from perfused rat liver and plated at 1×10⁵ cells per well into BIOCOAT 24-well (Bectin Dickinson Cat. No. 354408) plates in 1 mL Williams E medium supplemented with 10% Fetal bovine serum (FBS), Pen/Strep, glutamine and ITS+Premix (DB Biosciences, Cat. No. 354352). After four hours, cell culture medium was changed with 1 mL Williams E medium containing Pen/Strep, glutamine and ITS+Premix and the cells were incubated overnight under normal growth condition.

The following day, in the case of reporter transfection, for each transfection, 0.4 μg of clone 102-SEAP was mixed with the EFFECTENE reagent DNA-condensation buffer, Buffer EC, in a tube to a total volume of 350 μL. This was followed by adding 3.2 μL Enhancer and mixing by vortexing for 1 second. In the case of reporter/nuclear-receptor co-transfection, for each transfection, 0.3 μg of clone 102-SEAP and 0.1 μg hPXR (ATG)/pSGS or pCR3.1-hCAR was mixed with the Effectene reagent DNA-condensation buffer, Buffer EC, in a tube to a total volume of 350 μL. In a control transfection, 0.3 μg of clone 102-SEAP and 0.1 μg of pUC19 were cotransfected. The cotransfections were followed by adding 3.2 μL Enhancer and mixing by vortexing for 1 second. The transfections/cotransfections were incubated at room temperature for 5 minutes. Then, the mixtures were centrifuged for a few seconds to remove drops from the top of the tube. Next, to each of the mixtures, five μL of Effectene Transfection Reagent was added and the mixtures mixed by vortexing for 10 seconds. The mixtures were then incubated for ten minutes at room temperature to allow transfection complex formation.

Meanwhile, the growth medium for each of the wells of the 24-well plate containing the cells was removed from the wells and the cells in the wells washed once with 1 mL of growth medium. Then, 350 μL of fresh growth medium was added to each of the wells and 350 μL of growth medium was added to each of the tubes of transfection complexes, which were then mixed and immediately added drop-wise onto the cells in the well. The plates were gently swirled to ensure uniform distribution of the transfection complexes over the cells. The cells were incubated with the transfection media under normal growth conditions for six hours. Afterwards, the media containing the transfection complexes were removed and replaced it with 1 mL of Williams E medium containing antibiotics, glutamine, ITS, and either 10 μM Rifampicin, 1 μM CITCO (6-(4-Chlorophenyl)imidazo[2,1-b][1,3]thiazole-5-carbaldehyde O-(3,4-dichlorobenzyl)oxime), 1 mM Phenobarbital or DMSO alone. The cells were incubated for 48 hours at 37° C. The media were removed from the cells and assayed for SEAP activity using the Tropix Phospha-Light system kit.

In the control reaction, Clone 102-SEAP DNA and pUC19 DNA had been transfected into fresh rat hepatocytes to determine whether the clone 102-SEAP responded (and whether the SEAP signal could be measured in the media) to compounds recruiting the endogenous rat CAR or PXR in the absence of a co-transfected nuclear receptor. As shown in FIG. 14, there was no induction in the presence of any analyte tested. While in the case of Rifampicin this was expected because Rifampicin is not an inducer of CAR in the rat, no response was elicited by Phenobarbital, which is know to be an inducer of mouse CAR. The induction potential of CITCO in rodents is not believed to be known. It is expected that repeating the experiment with known strong CAR inducers such as TCPOBOP (1,4-bis[2-(3,5-dichloropyridyloxy)]benzene), which induces rat CAR, and, under conditions which ensure a consistent level of transfection efficiency would produce similar results. Thus, the results show that background expression of clone 102-SEAP in the rat hepatocytes was low to insignificant.

When the rat hepatocytes were cotransfected with clone 102-SEAP DNA and DNA encoding hCAR or hPXR, there was a significant induction in activation of the reporter gene expression in the presence of known CAR or PXR inducers and the induction was specific to the receptor. As shown in FIG. 14, Rifampicin induced expression via hPXR about 2.6 fold without inducing expression via hCAR, Phenobarbital induced expression via hCAR about 4 fold and expression via HPXR about 3.4 fold, and CITCO induced expression via hCAR about 4.2 fold without inducing expression via hPXR. The results indicate that the co-transfection protocol was successful and that the expression of clone 102-SEAP was driven solely by the interaction of the analyte and the transfected hCAR or HPXR. There was no apparent cross-reactivity between PXR inducers and hCAR or CAR inducers and hPXR. Moreover, hCAR appears to be regulated in the rat hepatocytes as it is regulated in the human liver. That is, unlike in most cell lines, in the cotransfected rat hepatocytes, the hCAR is not constitutively activated but instead is inducible. This is similar to the inducibility of hPXR in human liver, in HepG2, and in rat hepatocytes. While the results were strongly dependent on the condition of the hepatocytes and consequently on the transfection efficiency, the results demonstrated that the rat hepatocytes cotransfected with clone 102-SEAP DNA and an expression vector encoding the human CAR or PXR provide a sensitive assay for distinguishing analytes which activate either CAR or PXR or both.

The results demonstrate that rat hepatocytes cotransfected with a reporter gene operably linked to a composite promoter having a strength similar to or greater than that of the composite promoter of clone 102-SEAP and a gene expression cassette encoding hCAR provides a means for testing analytes solely for their potential for inducing hCAR activity. The results further demonstrate that rat hepatocytes cotransfected with a reporter gene operably linked to a composite promoter as above and a gene expression cassette encoding hPXR provides a means for testing analytes solely for their potential for inducing hPXR activity.

### EXAMPLE 7

In this example, clone 102-SEAP DNA was transfected into fresh human hepatocytes using the method of Example 6. The transfected cells were then incubated in media containing DMSO or 10 μM Rifampicin, 5 μM Clotrimazole, 10 μM TPP, 250 μM CITCO, or 50 μM Artemisinin for about 48 hours at 37° C. and then assayed for SEAP activity using the Tropix Phospha-Light system kit. The results are shown in FIG. 15 and show that all five analytes were inducers of expression of the reporter gene.

These results demonstrate that clone 102-SEAP DNA can be transfected into human hepatocytes and can be used to assess ability of analytes to induce CYP3A4 activity via CAR or PXR. This example shows that assaying for CYP3A4 activity via CAR or PXR using fresh human hepatocytes transfected only with clone 102-SEAP or a reporter gene operably linked to a composite promoter of similar to or greater strength than the composite promoter of clone 102-SEAP, is an improvement over prior art assays which require cotransfecting the reporter with a second vector encoding PXR or CAR.

### EXAMPLE 8

This example provides an efficient and quick method for transfecting HepG2 cells for use in assays of the present invention performed in a multiple well format. The advantage of the method is that because it is an in-liquid batch transfection, cells can be transfected in suspension and then plated in the wells of a multiple-well plate. The method is an improvement over two alternative but longer and less efficient methods: transfecting each well separately (very cumbersome and not reproducible for one or more 96 plates) and transfecting in a large (e.g., 15 cm or flask) tissue culture dish and splitting and re-plating the transfected cells into the appropriate sized container, e.g., 96-well tissue culture plates.

HepG2 cells are grown in tissue culture dishes to about 80% confluence. The medium is removed and the cells washed with PBS. The cells are trypsinized with a trypsin-EDTA solution. When the cells begin to detach, complete DMEM is added to the cells to block the trypsin and the cells transferred to centrifuge tubes. The cells are centrifuged at 1200 rpm for about 3-5 minutes. The supernatant fraction is removed and the cells resuspended in medium, about 15 to 20 mL for each 15 cm plate of cells harvested. A suitable medium is Hybridoma-SFM (GIBCO, Cat. No. 12045-085) or Opti-MEM (GIBCO, Cat. No. 51985-026); however, Hybridoma-SFM is preferred. The resuspended cells are centrifuges at 1200 rpm for about 3-5 minutes and resuspended in the above medium at about 15 to 20 mL cells for every 15 cm plate harvested. The cell clumps are broken up using a 20 mL syringe (1.2×40 gauge) and taking up and expelling the cells about 2 times. The cell suspension is passed through a Cell Strainer 70 μm (Falcon Cat. No. 35-2350) and the suspension diluted to about 500,000 cells per mL.

Preparation of transfection complex for a 96-well plate is as follows. The method can be adjusted to fit other multiple-well formats. A first mixture, MixA, is prepared by adding 4.5 μg (clone 102-SEAP DNA or reporter gene operably linked to another composite promoter or native promoter) and 0.5 μg hPXR (ATG)/pSGS to 500 μL of Hybridoma-SFM. Then 30 μL of PLUS Reagent (Life Technologies, cat. # 11514-015) is added and the mixture incubated for 15 minutes at room temperature.

A second mixture, MixB, is prepared by adding 20 μL of Lipofectamine (Life Technologies, Cat. No. 18324-020) to 500 μL of Hybridoma-SFM.

In a 15 mL polypropylene Falcon tube, MixA and MixB are combined and then 4 mL of cells at about 500,000 cells per mL are added to make a transfection mixture. The transfection mixture is gently mixed and then incubated 15 minutes at room temperature. Periodically, the tube is inverted to keep the cells in suspension.

Afterwards, to each well, a 50 μL aliquot of the transfection mixture is added. This results in about 20,000 cells per well. The cells are incubated at least three hours at 37° C. to allow cells to attach and for the transfection to progress. After about three hours, the transfection mixture is removed and replaced with 100 μL DMEM-GM medium with or without DMSO or analytes. After 48 hours at 37° C., the medium is removed and assayed for induction of hPXR activity.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1: A method for determining whether an analyte is capable of inducing expression of CYP3A4, which comprises:
(a) providing a cell comprising a nucleic acid, which includes two or more pregnane X receptor (PXR) binding sites in tandem operably linked to a heterologous promoter operably linked to a reporter gene;
(b) incubating the cell in a medium containing the analyte; and
(c) measuring expression of the reporter gene wherein an increase of the expression of the reporter gene in the presence of the analyte indicates that the analyte is capable of inducing expression of the CYP3A4.

2: The method of claim 1 wherein the PXR binding sites are selected from the group consisting of dDR3, dER6, and pER6.

3: The method of claim 2 wherein the dDR3 comprises the nucleotide sequence of SEQ ID NO:4, the dER6 comprises the nucleotide sequence of SEQ ID NO:7, and the pER6 comprises the nucleotide sequence of SEQ ID NO:1.

4: The method of claim 2 wherein the nucleic acid comprises at least one of the dER6 binding sites and at least one of the dDR3 binding sites.

5: The method of claim 1 wherein the cell expresses an endogenous PXR.

6: The method of claim 1 wherein the cell expresses an endogenous RXR.

7: The method of claim 1 wherein the cell is HepG2.

8: The method of claim 1 wherein the reporter is secreted embryonic alkaline phosphatase (SEAP).

9: The method of claim 1 wherein the two or more PXR binding sites in tandem comprise the nucleotide sequence of SEQ ID NO:18.

10-18. (canceled)

19. A method for determining whether an analyte is capable of inducing expression of CYP3A4, which comprises:
(a) providing a primary culture of hepatocyte cells comprising a first nucleic acid, which includes two or more pregnane X receptor (PXR) binding sites in tandem operably linked to a heterologous promoter operably linked to a reporter gene, and a second nucleic acid encoding the PXR;
(b) incubating the culture in a medium containing the analyte; and
(c) measuring expression of the reporter gene wherein an increase of the expression of the reporter gene in the presence of the analyte indicates that the analyte is capable of inducing expression of the CYP3A4.

20-22. (canceled)

23: The method of claim 19 wherein the hepatocyte cells are selected from the group consisting of rat, mouse, and human hepatocyte cells.

24: The method of claim 23 wherein the hepatocyte cells are rat hepatocyte cells.

25: The method of claim 19 wherein the PXR binding sites are selected from the group consisting of dDR3, dER6, and pER6.

26: The method of claim 19 wherein the dDR3 comprises the nucleotide sequence of SEQ ID NO:4, the dER6 comprises the nucleotide sequence of SEQ ID NO:7, and the pER6 comprises the nucleotide sequence of SEQ ID NO:1.

27: The method of claim 19 wherein the two or more PXR binding sites in tandem comprise the nucleotide sequence of SEQ ID NO:18.

28: A method for determining whether an analyte is capable of inducing expression of CYP3A4, which comprises:
(a) providing a primary culture of hepatocyte cells comprising a nucleic acid, which includes two or more pregnane X receptor (PXR) binding sites in tandem operably linked to a heterologous promoter operably linked to a reporter gene;
(b) incubating the culture in a medium containing the analyte; and
(c) measuring expression of the reporter gene wherein an increase of the expression of the reporter gene in the presence of the analyte indicates that the analyte is capable of inducing expression of the CYP3A4.

29: The method claim 28 wherein the PXR binding sites are selected from the group consisting of dDR3, dER6, and pER6.

30: The method of claim 28 wherein the dDR3 comprises the nucleotide sequence of SEQ ID NO:4, the dER6 comprises the nucleotide sequence of SEQ ID NO:7, and the pER6 comprises the nucleotide sequence of SEQ ID NO:1.

31: The method of claim 28 wherein the dDR3 comprises the nucleotide sequence of SEQ ID NO:5, the dER6 comprises the nucleotide sequence of SEQ ID NO:8, and the pER6 comprises the nucleotide sequence of SEQ ID NO:2.

32: The method of claim 28 wherein the dDR3 comprises the nucleotide sequence of SEQ ID NO:11, the dER6 comprises the nucleotide sequence of SEQ ID NO:12, and the pER6 comprises the nucleotide sequence of SEQ ID NO:10.

33: The method of claim 28 wherein the two or more PXR binding sites in tandem comprise the nucleotide sequence of SEQ ID NO:18.
